(19) United States
(12) Patent Application Publication
(10) Pub. No.: US 2007/0071755 A1
(43) Pub. Date: Mar. 29, 2007

(54) Novel nucleolar GTPases and method for controlling proliferation of cells

(76) Inventors: David Balasundaram, Singapore (SG); Du Xianming, Singapore (SG); Sundarasamy Mahalingam, Hyderabad (IN)

Correspondence Address: MORGAN LEWIS & BOCKIUS LLP, 1111 PENNSYLVANIA AVENUE NW, WASHINGTON, DC, 20004, US

(21) Appl. No.: 11/235,337

(22) Filed: Sep. 27, 2005

(51) Int. Cl. A61K 39/395; C12Q 1/68; G01N 33/574; C12N 9/16; C07H 21/04; C12P 21/06

(52) U.S. Cl. 424/155.1; 536/23.2; 435/69.1; 435/6; 435/7.23; 435/196; 435/320.1; 435/325

(57) ABSTRACT

There is provided an isolated polypeptide comprising an amino acid sequence at least 85% homologous to SEQ ID NO: 2 or SEQ ID N: 4, or a conservative variant thereof, wherein the polypeptide regulates proliferation of a cell. There is also provided an isolated polynucleotide encoding the polypeptide of the invention. There is also provided a method for inhibiting the proliferation of a cell, comprising altering the level of a polypeptide comprising an amino acid sequence at least 85% homologous to SEQ ID NO: 2 or SEQ ID NO: 4 in the cell, thereby inhibiting proliferation of the cell.

### FIELD OF INVENTION

This application relates to the field of cell cycle control. In particular, this invention relates to methods of inhibiting cell proliferation by altering the activities of novel nucleolar GTPases.

### BACKGROUND OF THE INVENTION

Cancer of various kinds accounts for a substantial proportion of human deaths. Cancer is an abnormal state in which uncontrolled proliferation of one or more cell populations in organs and tissues interferes with normal biological functioning. In the latter stages of cancer development, the proliferative changes are usually accompanied by other changes in cellular properties, including reversion to a less differentiated, more developmentally primitive state. These metastatic cancer cells may spread to other organs and cause pathology as well. When studied in the laboratory as cell or tissue culture, this in vitro correlate of cancer is called cellular transformation.

Patents in methods to control the proliferation of cancerous cells have been sought. A recent example is that for nucleostemin (see WO 2004/031731 A2). Nucleostemin (NS) is expressed preferentially in the nucleoli of central nervous system cells, embryonic stem cells, and several cancer cell lines. NS is thought to play a role in the development and control of stem and cancer cell proliferation.

While the provision of NS and the control of NS expression may be useful is the treatment of cancer, there is a need in this field of technique of new and alternative prophylactic or therapeutic treatments for cancer which may be substitutive, complementary or alternative to the putative use of NS.

### SUMMARY OF THE INVENTION

The present invention addresses the problems above, and in particular to provide new polypeptides, polynucleotides and methods to inhibit cell proliferation.

According to a first aspect, the present invention provides an isolated polypeptide comprising an amino acid sequence at least 85% homologous to SEQ ID NO: 2, wherein the polypeptide regulates proliferation of a cell. The isolated polypeptide may comprise an amino acid sequence at least 90%, 95% or 99% homologous to SEQ ID NO: 2 or is a conservative variant thereof. In particular, the isolated polypeptide comprises the amino acid sequence of SEQ ID NO: 2. There is also provided an isolated polypeptide consist of the amino acid sequence of SEQ ID NO:2. The invention also provides an isolated polynucleotide encoding the polypeptide according to the invention. In particular, there is provided a polynucleotide encoding a polypeptide comprising an amino acid sequence at least 85% homologous to SEQ ID NO: 2. According to a particular aspect, the isolated polynucleotide comprises or consists of the nucleotide sequence of SEQ ID NO: 1.

According to another aspect, the present invention provides an isolated polypeptide comprising an amino acid sequence at least 85% homologous to SEQ ID NO: 4, wherein the polypeptide regulates proliferation of a cell. The isolated polypeptide may comprise an amino acid sequence at least 90%, 95% or 99% homologous to SEQ ID NO: 4 or is a conservative variant thereof. In particular, the isolated polypeptide comprises the amino acid sequence of SEQ ID NO: 4. According to another aspect, the isolated polypeptide consist of the amino acid sequence of SEQ ID NO: 4. The invention also provides an isolated polynucleotide encoding the polypeptide according to the invention. In particular, there is provided a polynucleotide encoding a polypeptide comprising an amino acid sequence at least 85% homologous to SEQ ID NO: 4. In particular, there is also provided an isolated polynucleotide comprising or consisting of the nucleotide sequence of SEQ ID NO: 3.

The present invention provides an expression vector comprising the isolated polynucleotide of SEQ ID NO: 1 or SEQ ID NO: 3. The expression vector may be a viral vector.

According to another aspect, the isolated polynucleotides of the present invention may be degenerate variants thereof. The isolated polynucleotides of the present invention may also be operably linked to promoters.

The present invention also provides an isolated host cell transfected with the polynucleotide and/or the vector according to any aspect of the present invention. The isolated host cell may be an eukaryotic or prokaryotic cell.

There is also provided a method for producing a polypeptide comprising an amino acid sequence at least 85% homologous to SEQ ID NO: 2 or to SEQ ID NO:4, comprising transfecting an host cell with a polynucleotide encoding for a polypeptide comprising an amino acid sequence at least 85% homologous to SEQ ID NO: 2 or to SEQ ID NO: 4 or with a vector comprising a polynucleotide encoding for a polypeptide comprising an amino acid sequence at least 85% homologous to SEQ ID NO: 2 or to SEQ ID NO:4, and culturing the host cell. The method further comprises the step of isolating and/or purifying the expressed polypeptide.

The present invention also provides a method for inhibiting proliferation of a cell, comprising altering the level of a polypeptide comprising an amino acid sequence at least 85% homologous to SEQ ID NO: 2 in the cell, thereby inhibiting proliferation of the cell. The altering of the level of the polypeptide may further comprise decreasing the level of the polypeptide. The cell may further be a tumor cell or stem cell and the cell may either be in vitro or in vivo.

The method for inhibiting proliferation of a cell of the present invention may comprising altering the level of the polypeptides. For example, it may further comprise decreasing the transcription of nucleic acid sequences encoding the polypeptides. The altering the level of the polypeptide may also comprise use of a small inhibitory RNA (siRNA) that specifically binds a polynucleotide encoding the polypeptide. The small inhibitory RNA may be transcribed outside the cell and subsequently introduced into the cell. Alternatively, the small inhibitory RNA is encoded in an expression plasmid introduced into the cell wherein the small inhibitory RNA is subsequently transcribed in the cell.

According to another aspect, the present invention provides an antibody or fragment thereof that specifically binds the polypeptides comprising amino acid sequences at least 85% homologous to SEQ ID NO: 2 or to SEQ ID NO: 4. The antibody (or fragment thereof) may be selected from the group consisting of a monoclonal antibody and a polyclonal antibody (or fragments thereof). Further, the antibody may be comprised in a diagnostic kit, the kit further comprising information pertaining to the antibody.

According to another aspect, the present invention provides a method of screening agents that affect cell proliferation, the method comprising contacting candidate agents with at least one polypeptide having an amino acid sequence at least 85% homologous to the amino acid sequence of SEQ ID NO: 2 or 4, and evaluating the binding of the contacting against controls.

### BRIEF DESCRIPTION OF THE FIGURES

FIG. 1.

Grn1p is member of a unique family of MMR_HSR1-nucleolar GTPases with a highly conserved circularly permuted ‘G’-domain. (A) Schematic representation of three types of G-proteins illustrating the relative positions of the motifs that makeup the G-domain. The top bar represents GTPases with a circular permutation of the classic G-domain. The middle bar represents a small group of GTPases that belong to the Nog-subfamily. The bottom bar represents the classic G-proteins as exemplified by the Ras, EF-2 and heterotrimeric G-protein families. In the figure, RD=Regulatory domain (NLS, Basic domain); CC=Coiled-coil domain; G1=GxxxxGK(S/T), P-LOOP; G2=Effector (YAFTT or Switch I) or GxT(G2*); G3=DXXG/DXPG, (Switch II); G4=NKXD; G5=EXSAX or DARDP (G5*); and RG=Putative RNA-Binding domain. (B) Alignment of the above circularly permuted G-domain showing individual motifs G5*, G4, G1, G2*, G3 and the putative RNA-binding domain (RG). Motifs G5* and G2* correspond to G5-like and G2-like respectively. Representatives were chosen from a broad selection of eukaryotes S. pombe (Sp), S. cerevisiae (Sc), Human (Hs), Drosophila melanogaster (Dm), Danio rerio (Dm) and Arabidopsis thialiana (At).

Identical residues are shaded black (conservative substitutions are not indicated). Numbers to the left of each motif indicate the beginning of the amino acid motif. (C and D) 3-D representation of the highly conserved circularly permuted GTPases based on the structure of B. subtilis Ylqf GTPase as the consensus for the MMR_HSR₁ GTP-binding domain from the 3D-structure Entrez database, MMDB (http://www.ncbi.nlm.nih.gov/Structure/MMDB/mmdb.shtml). Images were visualized and recorded using NCBI's Cn3D4.1 software. G-motifs and the RG-domain are indicated with white arrows. Flat arrows represent β-sheets whereas cylinders represent α-helices.

FIG. 2.

Grn1 is required for wild type growth and encodes a nucleolar protein. (A) Grn1 (SPBC26H8.08c) was deleted as described in the text. Tetrad dissection yielded two fast-growing (wild type) and two slow-growing (null mutant) colonies. (B) A null mutant expressing full-length Grn1p:GFP (YNB544) or an empty vector (YNB546) were in EMM-leu medium. Optical density (OD₅₉₅) was determined at the indicated time points. (C) YNB544 (see above) was employed to show the localization of Grn1p. Nuclear DNA was stained with DAPI. Nucleoli were revealed by indirect immunostaining with anti-Fibrillarin (abcam, Cambridge, UK). Independent or merged images are indicated. (D) Wild type (YNB483) and null mutant (YNB484) strains were stained with DAPI and Aniline blue for visualizing the nucleus and septum respectively. Arrows indicate the septum. Bar indicates 10 microns.

FIG. 3.

Effect of Grn1p and GNL3L on processing of 35S pre-rRNA species. (A) Grn1:FLAG (YNB859) and GNL3L:FLAG (YNB858) were tested for genomically expressed FLAG-tagged Grn1p and GNL3L by western analysis and probing with anti-FLAG. The null mutant (YNB484) was used as control. (B) Pre-ribosomal RNA and mature rRNA species were detected in the above strains by northern hybridisational analysis. DNA probes specific for 5′ETS, 5.8S, ITS1 or ITS2 are indicated by bars under the respective flanks. The rRNA processing pathway was adapted from Good et al., 1997. Downward pointed arrows indicate relative positions of processing sites.

FIG. 4.

Rpl25a localization in ΔGrn1, Grn1-FLAG and ΔGrn1::GNL3L-FLAG strains. The null mutant (YNB484), Grn1:FLAG (YNB859) and GNL3L:FLAG (YNB858) were transformed with nmt1:Rpl25a:GFP (BNB221) to give YNB631, YNB1076 and YNB1075 respectively. GFP-fluorescence was visually inspected in >100 cells for each of the indicated strains. In >90% null mutant cells (YNB631) Rpl25a:GFP appeared inside nucleus with a significant accumulation within the nucleolus. For YNB1075 and YNB1076,>90% showed localization to the nuclear rim with no accumulation within the nucleolus. A representative image of each strain is depicted. The top right panel depicts the GFP and DAPI images of a single nucleus (indicated by arrowhead) that were enlarged and digitally manipulated to convert the GFP-green fluorescence to red.

FIG. 5.

The G-domain and RG-domain of Grn1p are required for growth. (A) The growth of all the indicated strains-WT (YNB544), grn1Δ(YNB546), ARG (YNB568), ΔG3 (YNB956), ΔG1 (YNB545), ΔG4 (YNB611), ΔG5 (YNB566) and ΔCC (YNB567) was determined. Strains were struck for single colonies on EMM-leu plates with (nmt1 OFF) or without 15 μM thiamine (nmt1 ON). (B) The total proteins of all the strains were isolated and processed by western using anti-GFP antibody. (C) GFP, Grn1:GFP, ΔG5-Grn1:GFP, ΔG4-Grn1:GFP, ΔG1-Grn1:GFP, AG3-Grn1:GFP, ARG-Grn1:GFP, from pBNB340, pBNB338, pBNB335, pBNB336, pBNB337, pBNB417 and pBNB339 respectively (Table 2), were transcribed and translated in the presence of L-[35S]methionine using a TNT-coupled Reticulocyte Lysate System (Promega, Madison, Wis.) according to the manufacturer's instructions. One microliter of each of product was analyzed on a 12% SDS-PAGE gel and exposed to X-ray film.

FIG. 6.

Effect of deletions in the G-domain and RG-domain on the localization of Grn1p. (A) Strains indicated in FIG. 5A with plasmids containing GFP-tagged mutant versions of Grn1p expressed from an nmt1 inducible promoter were grown in EMM-leu medium with and without 15 μM thiamine. Only cells that were induced (nmt1 ON) are shown. Bar indicates 10 microns. (B). The GFP and DAPI images of a single nucleus from ΔG5 (YNB566) and ΔCC (YNB567) marked with colored arrowheads, were enlarged and digitally manipulated to convert the GFP-green fluorescence to red in order to render a sharper contrast against the blue DAPI fluorescence. This more vividly delineates the nucleolar region from the extra-nucleolar region. (C) Cartoon shows a single nucleus with morphological subcompartments of the nucleolus. FC is the fibrillar center, DFC denotes the dense fibrillar component and GC represents the granular component. White arrow indicates the accumulation of GFP-signal at the granular component on the nucleolus.

FIG. 7.

Expression of a human gene GNL3L rescues the growth defect of the null mutant. (A) The legend for the strains and inserts is as follows: 1. YNB1003 (ScNug1p); 2. YNB961 (HsNS); 3. YNB805 (GNL3L); 4. YNB795(Ngp1); 5. YNB544 (Wild type Grn1); and 6. YNB546 (empty vector). GNL3L-GFP (92.4 KDa), Grn1p-GFP (80.2 KDa) and GFP (26.8 KDa) are indicated by arrows. (B) Growth of GNL31:FLAG (YNB858) and Grn1:FLAG (YNB859) is compared with a null mutant (YNB484) in YES medium. (C) GNL3L and Grn1p co-localize with nucleolin in Cos-7 cells. Localization of GNL3L and Grn1p was determined by confocal microscopy. Nucleoli were revealed by immunostaining with anti-Nucleolin. (D) S. pombe cells showing localization of GNL3L:GFP in S. pombe. Wild type S. pombe was transformed with an expression vector containing GNL3L:FLAG (BNB395). Nucleoli were revealed by immunostaining with anti-fibrillarin and GNL3L with anti-FLAG. Independent or merged images are indicated. Bar indicates 4 microns.

FIG. 8.

siRNA knockdown of GNL3L in HeLa cells. (A) Cultures of HeLa cells were tranfected with the indicated siRNA sequence. After 24 h post transfection, the siRNA expression cells were selected in the presence of Neomycin (500 ug/ml) for 120 h and photographed (B) RT-PCR analysis of GNL3L transcript. Total RNA was isolated from the cells tranfected with the indicated siRNA. RT-PCR analysis was performed as described in Materials and Methods. β-actin was used as internal control.

FIG. 9

Effect of deleting the putative nucleolar/nuclear targeting domain on Grn1:GFP localization and on growth. (A) Alignment of the N-terminal domains of GNL3L, Grn1p and NS showing the remarkably conserved pattern of basic residues. The indicated amino acids within Grn1p sequence NLS1Δ, AA6-22 and NLSΔΔ, AA6-36 were deleted. (B) Strains containing the above deletions NLS1Δ, AA6-22 (YNB592), NLSΔΔ, AA6-36 (YNB593) and the wild type Grn1 (YNB591) (Table 2) were grown in EMM-leu medium and examined for GFP-fluorescence. (C) Growth of YNB592 and YNB593 were compared with the wild type strain, YNB591 on EMM-leu medium. (D) Equal amounts of cells from the indicated strains were subjected to western analysis and probed with anti-GFP.

FIG. 10

Alignment of the GTPases GLN3L. Grn1p and Nucleostemin (NS). Identical residues are shaded black (conservative substitutions are not included).

FIG. 11

FIG. 11 is the Blastp polypeptide sequence comparison between GNL3L and NS showing no significant similarity between these two sequences.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

Definitions

Unless otherwise noted, the technical terms used herein are according to conventional usage in the field of biotechnology and understood by persons skilled in this art. Definitions of common terms in molecular biology may be found in standard texts such as Benjamin Lewin, Genes V, published by Oxford University Press, 1994 (ISBN 0-19-854287-9); Kendrew et al. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various aspects of the present invention, the following explanations of specific technical terms are provided. These explanations were compiled and/or paraphrased from the above texts as well as from other publications in the public domain. In the explanations hereunder, the term “polypeptide of the invention” refers to either one or both of the proteins Grn1p and GNL3L.

Agent: Any polypeptide, compound, small molecule, organic compound, salt, polynucleotide or other molecule of interest.

Alter: A change in an effective amount of a substance of interest, such as a polynucleotide or polypeptide. The amount of the substance can changed by a difference in the amount of the substance produced, by a difference in the amount of the substance that has a desired function, or by a difference in the activation of the substance. The change can be an increase or a decrease. The alteration can be in vivo or in vitro. In several aspects, altering an effective amount of a polypeptide or polynucleotide is at least about a 50%, 60%, 70%, 85%, 90%, 95%, 96%, 97%, 98%, 99%, or 100% increase or decrease in the effective amount (level) of a substance. In another aspect, an alteration in polypeptide or polynucleotide affects a physiological property of a cell, such as the differentiation, proliferation, or senescence of the cell.

Animal: Living multi-cellular vertebrate organisms, a category that includes, for example, mammals and birds. The term mammal includes both human and non-human mammals. Similarly, the term “subject” includes both human and veterinary subjects.

Antibiotic Resistance Cassette: A nucleic acid sequence encoding one or more selectable markers which confer resistance to that antibiotic in a host cell in which the nucleic acid is translated. Examples of antibiotic resistance cassettes include, but are not limited to kanamycin, ampicillin, tetracycline, chloramphenicol, neomycin, hygromycin, and zeocin.

Antisense, Sense, and Antigene: DNA has two strands, a 5′ to 3′strand, referred to as the plus strand, and a 3′ to 5′ strand, referred to as the minus strand.

Because RNA polymerase adds nucleic acids in a 5′ to 3′ direction, the minus strand of the DNA serves as the template for the RNA during transcription. Thus, the RNA formed will have a sequence complementary to the minus strand and identical to the plus strand (except that Uracil is substituted for Thymine).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a DNA target. An antisense RNA (as RNA) is a molecule of RNA complementary to a sense (encoding) nucleic acid molecule. cDNA (complementary DNA): A piece of DNA lacking internal, non-coding segments (introns) and regulatory sequences that determine transcription. cDNA is synthesized in the laboratory by reverse transcription from messenger RNA extracted from cells.

Degenerate variant: A polynucleotide encoding a polypeptide of the invention that includes a sequence that is degenerate as a result of the genetic code. There are 20 natural amino acids, most of which are specified by more than one codon. Therefore, all degenerate nucleotide sequences are included as long as the amino acid sequence of a polypeptide of the invention encoded by the nucleotide sequence is unchanged.

Differentiation: Refers to the process whereby relatively unspecialized cells (eg, embryonic cells) acquire specialized structural and/or functional features characteristic of mature cells. Similarly, “differentiate” also refers to this process.

Typically, during differentiation, cellular structure is altered and tissue-specific proteins appear.

Effective amount or therapeutically effective amount: The amount of agent sufficient to prevent, treat, reduce and/or ameliorate the symptoms and/or underlying causes of any of a disorder or disease. In one aspect, an effective amount is sufficient to reduce or eliminate a symptom of a disease. In another aspect, an effective amount is an amount sufficient to overcome the disease itself.

Embryonic stem (ES) cells: Pluripotent cells isolated from the inner cell mass of the developing blastocyst. ES cells can be derived from any organism, including mammals.

In one aspect, ES cells are produced from mammals such as mice, rats, rabbits, guinea pigs, goats, pigs, cows and humans. Human and murine derived ES cells are preferred. ES cells are totipotent cells, meaning that they can generate all of the cells present in the body (bone, muscle, brain cells, etc.).

Methods for producing murine ES cells can be found in U.S. Pat. No. 5,670,372, herein incorporated by reference. Methods for producing human ES cells can be found in U.S. Pat. No. 6,090,622, WO 00/70021 and WO 00/27995, herein incorporated by reference.

Enhancer: A cis-regulatory sequence that can elevate levels of transcription of a coding sequence from an adjacent promoter. Many tissue specific enhancers can determine spatial patterns of gene expression in higher eukaryotes. Enhancers can act on promoters over many tens of kilobases of DNA and can be 5′ or 3′ to the promoter they regulate. Enhancers can function either by initiating transcription from a promoter operably linked to the enhancer or by providing binding sites for gene regulatory proteins that increase transcription of a minimal promoter.

Epitope: An antigenic determinant. These are particular chemical groups or peptide sequences on a molecule that are antigenic, i.e. that elicit a specific immune response. An antibody specifically binds a particular antigenic epitope on a polypeptide.

Expand: A process by which the number or amount of cells in a cell culture is increased due to cell division. Similarly, the terms “expansion” or “expanded” refers to this process. The terms “proliferate”, “proliferation” or “proliferated” may be used interchangeably with the words “expand”, “expansion” or “expanded.” Typically, during an expansion phase, the cells do not differentiate to form mature cells.

GNL3L: A polypeptide having an amino acid sequence at least 85% identity to SEQ ID NO: 2 which affects the proliferation of a cell. In one aspect, a GNL3L has the amino acid sequence indicated in SEQ ID NO: 2.

Grn1p: A polypeptide having an amino acid sequence at least 85% identity to SEQ ID NO: 4 which affects the differentiation and/or proliferation of a cell. In one aspect, a Grn1p has the amino acid sequence indicated in SEQ ID NO: 4.

Heterologous: A heterologous sequence is a sequence that is not normally (ie in the wild type sequence) found adjacent to a second sequence. In one aspect, the sequence is from a different genetic source, such as a virus or organism, from the second sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridization: The process wherein oligonucleotides and their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine (T)) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms which indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example, under physiological conditions in the case of in vivo assays.

Such binding is referred to as “specific hybridization.” Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ ion concentration) of the hybridization buffer will determine the stringency of hybridization.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (eg, SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. The parameters of salt concentration and temperature can be varied to achieve the optimal level of identity between the probe and the target nucleic acid.

Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of this disclosure, “stringent conditions” encompass conditions under which hybridization will only occur if there is less than 30% mismatch between the hybridization molecule and the target sequence. “Stringent conditions” may be broken down into particular levels of stringency for more precise definition.

Thus, as used herein, “moderate stringency” conditions are those under which molecules with more than 30% sequence mismatch will not hybridize; conditions of “medium stringency” are those under which molecules with more than 20% mismatch will not hybridize, and conditions of “high stringency” are those under which sequences with more than 10% mismatch will not hybridize.

Molecules with complementary nucleic acids form a stable duplex or triplex structure when the strands bind, or hybridize, to each other by forming Watson-Crick, Hoogsteen or reverse Hoogsteen base pairs. Stable binding occurs when an oligonucleotide remains detectably bound to a target nucleic acid sequence under the required conditions. “Complementarity” is the degree to which bases in one nucleic acid strand base pair with the bases in a second nucleic acid strand.

Complementarity is conveniently described by the percentage, ie, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between the oligonucleotide and the target sequence to achieve detectable binding, and disrupt expression of gene products (such as M-CSF). When expressed or measured by percentage of base pairs formed, the percentage complementarity that fulfills this goal can range from as little as about 50% complementarity to full (100%) complementary. In general, sufficient complementarity is at least about 50%. In one aspect, sufficient complementarity is at least about 75% complementarity. In another aspect, sufficient complementarity is at least about 90% or about 95% complementarity. In yet another aspect, sufficient complementarity is at least about 98% or 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol 100: 266-285, 1983, and by Sambrook et al. (ed.), Molecular Cloning: A Laboratory Manual, 2nd ed, vol. 1-3, Cold Spring Harbor Laboratory Press, Cold Spring Harbor, N.Y., 1989.

Interefering with or inhibiting (expression of a target gene): This phrase refers to the ability of a siRNA or other molecule to measurably reduce the expression of a target gene. It contemplates reduction of the end-product of the gene, eg, the expression or function of the encoded protein, and thus includes reduction in the amount or longevity of the mRNA transcript. It is understood that the phrase is relative, and does not require absolute suppression of the gene. Thus, in certain aspects, interfering with or inhibiting gene expression of a target gene requires that, following application of the dsRNA, the gene is expressed at least 5% less than prior to application of double-stranded RNA dsDNA, such as at least 10% less, at least 15% less, at least 20% less, at least 25% less, or even more reduced. Thus, in some particular aspects, application of a dsRNA reduces expression of the target gene by about 30%, about 40%, about 50%, about 60%, or more. In specific examples, where the dsRNA is particularly effective, expression is reduced by 70%, 85%, 85%, 90%, 95%, or even more.

In vitro amplification: Techniques that increase the number of copies of a nucleic acid molecule in a sample or specimen. An example of amplification is the polymerase chain reaction (PCR), in which a biological sample collected from a subject is contacted with a pair of oligonucleotide primers, under conditions that allow for the hybridization of the primers to nucleic acid template in the sample.

The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320308); gap filling ligase chain reaction amplification (see U.S. Pat. No. 5,427,930); coupled ligase detection and PCR (see U.S. Pat. No. 6,027,89); and NASBATM RNA transcription-free amplification (see U.S. Pat. No. 6,025,134).

Isolated: An “isolated” biological component (such as a nucleic acid, peptide or protein) has been substantially separated, produced apart from, or purified away from other biological components in the cell of the organism in which the component naturally occurs, ie, other chromosomal and extrachromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide, such as a Grn1p or GNL3L. Biologically functional fragments, for example, can vary in size from a polypeptide fragment as small as an epitope capable of binding an antibody molecule to a large polypeptide capable of participating in the characteristic induction or programming of phenotypic changes within a cell, including affecting cell proliferation or differentiation. An “epitope” is a region of a polypeptide capable of binding an immunoglobulin generated in response to contact with an antigen. Thus, smaller peptides containing the biological activity of insulin, or conservative variants of the insulin, are thus included as being of use. A conservative variant of a polypeptide is one that includes no more than fifty conservative amino acid substitutions of the polypeptide, such as no more than two, no more than five, no more than 10, or no more than 20 conservative amino acid substitutions in that polypeptide sequence.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one aspect, the polypeptide is at least 50%, for example at least 85% free of other proteins, lipids, carbohydrates or other materials with which it is naturally associated. In another aspect, the polypeptide is at least 90% free of other proteins, lipids, carbohydrates or other materials with which it is naturally associated. In yet another aspect, the polypeptide is at least 95% free of other proteins, lipids, carbohydrates or other materials with which it is naturally associated.

Conservative substitutions replace one amino acid with another amino acid that is similar in size, hydrophobicity, etc. Examples of conservative substitutions are shown below.

| Original Residue | Possible Conservative Substitution(s) |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Variations in the cDNA sequence that result in amino acid changes, whether conservative or not, should be minimized in order to preserve the functional and immunologic identity of the encoded protein. Thus, in several non-limiting examples, a polypeptide of the invention includes at most two, at most five, at most 10, at most 20, or at most 50 conservative substitutions. The immunologic identity of the protein may be assessed by determining whether it is recognized by an antibody; a variant that is recognized by such an antibody is immunologically conserved. Any cDNA sequence variant will preferably introduce no more than 20, and preferably fewer than 10 amino acid substitutions into the encoded polypeptide. Variant amino acid sequences may be, for example, at least 85%, 90% or even 95% or 98% identical to the native amino acid sequence.

Pharmaceutically acceptable carriers: The pharmaceutically acceptable carriers of use are conventional. Remington's Pharmaceutical Sciences, by E W Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Pharmaceutical agent: A chemical compound, small molecule, or other composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between six and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Primers: Short nucleic acids, for example, DNA oligonucleotides 10 nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, eg, by the polymerase chain reaction (PCR) or other nucleic-acid amplification methods known in the art.

Probes and primers as used herein may, for example, include at least 10 nucleotides of the nucleic acid sequences that are shown to encode specific proteins.

In order to enhance specificity, longer probes and primers may also be employed, such as probes and primers that comprise 15, 20, 30, 40, 50, 60, 70, 80, 90 or 100 consecutive nucleotides of the disclosed nucleic acid sequences.

Methods for preparing and using probes and primers are described in the references, for example Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences; Innis et al. (1990) PCR Protocols—A Guide to Methods and Applications, Knis et al. (Eds.), Academic Press, San Diego, Calif.

PCR primer pairs can be derived from a known sequence, for example, by using computer programs intended for that purpose such as Primer (Version 0.5, 1991, Whitehead Institute for Biomedical Research, Cambridge, Mass.).

When referring to a probe or primer, the term specific for (a target sequence) indicates that the probe or primer hybridizes under stringent conditions substantially only to the target sequence in a given sample comprising the target sequence.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, eg, by genetic engineering techniques. Similarly, a recombinant protein is one encoded by a recombinant nucleic acid molecule.

Senescence: The inability of a cell to divide further. A senescent cell is still viable, but does not divide.

Sequence identity: The similarity between amino acid sequences or between nucleic acid sequences can be expressed in terms of the percentage of conservation between the sequences, otherwise referred to as sequence similarity. Sequence identity is frequently measured in terms of percentage identity (or similarity or homology); the higher the percentage, the more similar the two sequences are.

Homologues or variants of a nucelotide or amino acid sequence will possess a relatively high degree of sequence identity or homology when aligned using standard methods. Methods of alignment of sequences for comparison are well known in the art.

The NCBI Basic Local Alignment Search Tool (BLAST) is available from several sources, including the National Center for Biotechnology Information (NCBI, Bethesda, Md.) and on the Internet, for use in connection with the sequence analysis programs blastp, blastn, blastx, tblastn and tblastx. A description of how to determine sequence identity using this program is available on the NCBI website on the Internet. Other specific, non-limiting examples of sequence alignment programs specifically designed to identify conserved regions of genomic DNA of greater than or equal to 100 nucleotides are PIPMaker (Schwartz et al, Genome Research 10: 577-586, 2000) and DOTTER (Erik et al., Gene 167: GC1-10, 1995).

Homologues and variants of a nucleotide or amino acid sequence are typically characterized by possession of at least 75%, for example at least 85%, 90%, 95%, 98%, or 99%, sequence identity counted over the full length alignment with the originating NS sequence using the NCBI Blast 2.0, set to default parameters. Methods for determining sequence identity over such short windows are available at the NCBI website on the Internet. One of skill in the art will appreciate that these sequence identity ranges are provided for guidance only; it is entirely possible that strongly significant homologues could be obtained that fall outside of the ranges provided.

Small inhibitory RNA (siRNA): Abbreviation for small inhibitory RNA, a short sequence of RNA which can be used to silence gene expression. In particular, it indicates double stranded RNAs (dsRNAs) that can induce gene-specific inhibition or interference of expression in invertebrate and vertebrate species. These RNAs are suitable for interference or inhibition of expression of a target gene and comprise double stranded RNAs of about 15 to about 40 nucleotides containing a 3′ and/or 5′overhang on each strand having a length of 0 to about five nucleotides, wherein the sequence of the double stranded RNAs is substantially identical to a portion of an mRNA or transcript of the target gene for which interference or inhibition of expression is desired. The double stranded RNAs can be formed from complementary ssRNAs or from a single stranded RNA that forms a hairpin or from expression from a DNA vector.

In addition to native RNA molecules, RNA suitable for inhibiting or interfering with the expression of a target sequence encoding a polypeptide of the invention includes RNA derivatives and analogs. For example, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′-O-alkylated residues or 2′-deoxy-2′-halogenated derivatives. Particular examples of such carbohydrate moieties include 2′-O-methyl ribosyl derivatives and 2′-O-fluoro ribosyl derivatives.

The RNA bases may also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

Stem cell: A cell that can generate a fully differentiated functional cell of more than one given cell type. The role of stem cells in vivo is to replace cells that are destroyed during the normal life of an animal. Generally, stem cells can divide without limit. After division, the stem cell may remain as a stem cell, become a precursor cell, or proceed to terminal differentiation. Although appearing morphologically unspecialized, the stem cell may be considered differentiated where the possibilities for further differentiation are limited. A precursor cell is a cell that can generate a fully differentiated functional cell of at least one given cell type.

Generally, precursor cells can divide. After division, a precursor cell can remain a precursor cell, or may proceed to terminal differentiation. In one specific, non-limiting example, a “pancreatic stem cell” is a stem cell of the pancreas. In one aspect, a pancreatic stem cell gives rise to all of the pancreatic endocrine cells, eg, the α cells, β cells, δ cells, and pancreatic precursor cells, but does not give rise to other cells such as the pancreatic exocrine cells. A “pancreatic precursor cell” is a precursor cell of the pancreas. In one aspect, a pancreatic precursor cell gives rise to more than one type of pancreatic endocrine cell. One specific, non-limiting example of a pancreatic precursor cell is a cell that give rise to α and β cells.

Subject: Any mammal, such as humans, non-human primates, pigs, sheep, cows, rodents and the like, which is to be the recipient of the particular treatment. In one aspect, a subject is a human subject or a murine subject.

Therapeutic agent: Used in a generic sense, it includes treating agents, prophylactic agents, and replacement agents.

Transduced and transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as chemical methods (eg, calcium-phosphate transfection), physical methods (eg, electroporation, microinjection, particle bombardment), fusion (eg, liposomes), receptor-mediated endocytosis (eg, DNA-protein complexes, viral envelope/capsid-DNA complexes) and by biological infection by viruses such as recombinant viruses (see Wolff, J A, (ed.), Gene Therapeutics, Birkhauser, Boston, Mass., USA, 1994). In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is one indicia of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Unless otherwise explained, all technical and scientific terms used herein have the same meaning as commonly understood by one of ordinary skill in the art to which this disclosure belongs. The singular terms “a”, “an” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

Polypeptides and polynucleotides of the present invention. Substantially isolated and/or purified polypeptides of the invention are disclosed herein. In one aspect, a GNL3Lpolypeptide has a sequence at least 85% homologous to the amino acid sequence set forth in SEQ ID NO: 2, such as, but not limited to, at least 90%, 95%, or 99% homologous to the amino acid sequence set forth in SEQ ID NO: 2. Thus, in one specific example, a GNL3L polypeptide has a sequence set forth as SEQ ID NO: 2:

In another aspect, a GNL3L polypeptide has a sequence as set forth as SEQ ID NO: 2 or is a conservative variant of SEQ ID NO: 2, such that it includes no more than fifty conservative substitutions of SEQ ID NO: 2, such as no more than two, no more than five, no more than ten, or no more than twenty conservative amino acid substitutions in SEQ ID NO: 2. In another aspect, a Grn1p polypeptide has an amino acid sequence as set forth as SEQ ID NO: 2.

In one aspect, a Grn1p polypeptide has a sequence at least 85% homologous to the amino acid sequence set forth in SEQ ID NO: 4, such as, but not limited to, at least 90%, 95%, or 99% homologous to the amino acid sequence set forth in SEQ ID NO: 4. Thus, in one specific example, a Grn1p polypeptide has a sequence set forth as SEQ ID NO: 4.

In another aspect, a Grn1p polypeptide has a sequence as set forth as SEQ ID NO: 4 or is a conservative variant of SEQ ID NO: 4, such that it includes no more than fifty conservative substitutions of SEQ ID NO: 4, such as no more than two, no more than five, no more than ten, or no more than twenty conservative amino acid substitutions in SEQ ID NO: 4. In another aspect, a Grn1p polypeptide has an amino acid sequence as set forth as SEQ ID NO: 4.

Specific, non-limiting examples of a GNL3L polypeptide are conservative variants of SEQ ID NO: 2 and that for a Grn1p polypeptide are conservative variants of SEQ ID NO: 4. Examples of conservative substitutions is provided above. Substitutions of the amino acid sequences shown in SEQ ID NO: 2 or SEQ ID NO: 4 can be made based on this list of substitutions. Thus, one non-limiting example of a conservative variant is substitution of amino acid one (Met) of SEQ ID NO: 2 with an arginine residue. Using the sequence provided as SEQ ID NO: 2, and the description of conservative amino acid substitutions provided, one of skill in the art can readily ascertain sequences of conservative variants. In several aspects, a conservative variant includes at most one, at most two, at most five, at most ten, or at most fifteen conservative substitutions of the sequence shown in SEQ ID NO: 2.

Generally, a conservative variant will bind to antibodies that immunoreact with a polypeptide including a sequence set forth as SEQ ID NO: 2, and/or will immunoreact with a polypeptide including a sequence set forth as SEQ ID NO: 4.

Fragments and variants of a polypeptide can readily be prepared by one of skill in the art using molecular techniques. In one aspect, a fragment of a polypeptide of the invention includes at least eight, 10, 15, or 20 consecutive amino acids of the polypeptide. In another aspect, a fragment of a polypeptide of the invention includes a specific antigenic epitope found on a full-length polypeptide in question. In a further aspect, a fragment of a polypeptide is a fragment that confers a function of that polypeptide when transferred into a cell of interest, such as, but not limited to, inducing differentiation or decreasing proliferation of the cell.

One skilled in the art, given the disclosure herein, can purify any desired polypeptide using standard techniques for protein purification. The substantially pure polypeptide will yield a single major band on a non-reducing polyacrylamide gel. The purity of the polypeptide can also be determined by amino-terminal amino acid sequence analysis.

Minor modifications of the primary amino acid sequence of the polypeptides of the invention may result in peptides which have substantially equivalent activity as compared to the unmodified counterpart polypeptide described herein. Such modifications may be deliberate, as by site-directed mutagenesis, or may be spontaneous. All of the polypeptides produced by these modifications are included herein under the scope of the present invention.

One of skill in the art can readily produce fusion proteins including a first polypeptide of the invention with a second polypeptide of the invention. Optionally, a linker can be included between a first polypeptide of the invention and a second polypeptide of the invention. Fusion proteins include, but are not limited to, a polypeptide including a polypeptide of the invention and a marker protein. In one aspect, the marker protein can be used to identify or purify a polypeptide of the invention. Exemplary fusion proteins include, but are not limited to, green fluorescent protein (GFP), six histidine residues, or myc and a polypeptide of the invention.

As disclosed herein, an increase or decrease in the concentration of a polypeptide of the invention induces differentiation of cells, such as, but not limited to, stem cells. An increase or decrease in the concentration of a polypeptide of the invention inhibits proliferation of cells, such as, but not limited to, stem cells.

Polynucleotides encoding the polypeptides of the invention are also provided. These polynucleotides include DNA, cDNA and RNA sequences which encode the polypeptides of the invention. It is understood that all polynucleotides encoding a polypeptide of the invention are also included herein, as long as they encode a polypeptide with the recognized activity, such as the binding to an antibody that recognizes one of the polypeptides of the invention, or affecting cell proliferation. The polynucleotides include sequences that are degenerate as a result of the genetic code. There are 20 natural amino acids, most of which are specified by more than one codon. Therefore, all degenerate nucleotide sequences are included as long as the amino acid sequence of a polynucleotide of the invention encoded by the nucleotide sequence is functionally unchanged.

Another specific non-limiting example of a polynucleotide encoding a polypeptide according to any aspect of the invention. In particular, a polynucleotide encoding a polypeptide having at least 85% homology to SEQ ID NO: 2 or SEQ ID NO 4, such as a polypeptide at least 90%, 95%, or 99% homologous to SEQ ID NO: 2 or SEQ ID NO: 4. In particular, the polynucleotide according to the invention encodes a polypeptide having an antigenic epitope or function of the polypeptide according to any aspect of the invention. Yet another specific non-limiting example of a polynucleotide encoding a polypeptide of the invention is a polynucleotide that encodes a polypeptide that is specifically bound by an antibody that specifically binds the polypeptide comprising or having the amino acid sequence of SEQ ID NO: 2 or SEQ ID NO: 4.

According to a particular aspect, a polynucleotide according to the invention comprises or has the nucleotide sequence of SEQ ID NO:1. According to another aspect, a polynucleotide according to the invention comprises or has the nucleotide sequence of SEQ ID NO:3.

The polynucleotides of the invention include a recombinant DNA which may be incorporated into a vector; into an autonomously replicating plasmid or virus; or into the genomic DNA of a prokaryote or eukaryote, or which exists as a separate molecule (eg, a cDNA) independent of other sequences. The nucleotides can be ribonucleotides, deoxyribonucleotides, or modified forms of either nucleotide. The term includes single and double forms of DNA. Also included in this disclosure are fragments of the above-described nucleic acid sequences that are at least 15 bases in length, which is sufficient to permit the fragment to selectively hybridize to DNA that encodes the disclosed any of the polynucleotides of the invention (eg, a polynucleotide that encodes a polypeptide comprising or consisting of SEQ ID NO: 2 or SEQ ID NO: 4) under physiological conditions. The term “selectively Hybridize” refers to hybridization under moderately or highly stringent conditions, which excludes non-related nucleotide sequences.

Expression Systems: A polynucleotide encoding a polypeptide of the invention may be included in an expression vector to direct expression of the nucleic acid sequence coding for a polypeptide of the invention. Thus, other expression control sequences including appropriate promoters, enhancers, transcription terminators, a start codon (ie, ATG) in front of a protein-encoding gene, splicing signal for introns, maintenance of the correct reading frame of that gene to permit proper translation of mRNA, and stop codons can be included with a sequence coding for a polypeptide of the invention in an expression vector. Generally expression control sequences include a promoter, a minimal sequence sufficient to direct transcription.

The expression vector typically may contain an origin of replication, a promoter, as well as specific genes which allow phenotypic selection of the transformed cells (eg an antibiotic resistance cassette). Vectors suitable for use include, but are not limited, to the pMSXND expression vector for expression in mammalian cells. Generally, the expression vector will include a promoter. The promoter can be inducible or constitutive. The promoter can be tissue specific. Suitable promoters include the thymidine kinase promoter (TK), metallothionein 1, polyhedron, neuron specific enolase, thyrosine hyroxylase, beta-actin, or other promoters. In one aspect, the promoter is a heterologous promoter.

In one example, the polynucleotide encoding a polypeptide of the invention is located downstream of the desired promoter. Optionally, an enhancer element is also included, and can generally be located anywhere on the vector and still have an enhancing effect. However, the amount of increased activity will generally diminish with distance.

Expression vectors including a polynucleotide encoding a polypeptide of the invention can be used to transform host cells. Hosts can include isolated microbial, yeast, insect and mammalian cells, as well as cells located in the organism. Biologically functional viral and plasmid DNA vectors capable of expression and replication in a host are known in the art, and can be used to transfect any cell of interest. Where the cell is a mammalian cell, the genetic change is generally achieved by introduction of the DNA into the genome of the cell (ie, stable) or as an episome.

A “transfected cell” is a cell or host cell into which (or into an ancestor of which) has been introduced, by means of recombinant DNA techniques, a DNA molecule encoding a polypeptide of the invention. Transfection of a host cell with recombinant DNA may be carried out by conventional techniques as are well known to those skilled in the art.

Where the host is prokaryotic, such as E. coli, competent cells which are capable of DNA uptake can be prepared from cells harvested after exponential growth phase and subsequently treated by the CaCk method using procedures well known in the art. Alternatively, MgCl2 or RbCL can be used. Transformation can also be performed after forming a protoplast of the host cell if desired, or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding a polypeptide of the invention, and a second foreign DNA molecule encoding a selectable phenotype, such as neomycin resistance.

Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Other specific, non-limiting examples of viral vectors include adenoviral vectors, lentiviral vectors, retroviral vectors, and pseudorabies vectors.

The isolated polynucleotide sequences coding for a polypeptide of the invention disclosed herein can also be used in the production of transgenic animals such as transgenic mice, as described below.

In one aspect, a non-human animal is generated that carries a transgene comprising a nucleic acid encoding a polypeptide of the invention operably linked to a promoter.

Specific promoters of use include, but are not limited to, a tissue specific promoter such as, but not limited to, an immunoglobulin promoter, a neuronal specific promoter, or the insulin promoter. Specific promoters of use also include a constitutive promoter, such as, but not limited to, the thymdine kinase promoter or the human p-globin minimal, or an actin promoter, amongst others.

This construct may be introduced into a vector to produce a product that is then amplified, for example, by preparation in a bacterial vector, according to conventional methods (see, for example, Russel and Sambrook, Molecular Cloning: a Laboratory Manual, Cold Spring Harbor Press, 2001). The amplified construct is thereafter excised from the vector and purified for use in producing transgenic animals.

Any transgenic animal can be of use in the methods disclosed herein, provided the transgenic animal is a non-human animal. A “non-human animal” includes, but is not limited to, a non-human primate, a farm animal such as swine, cattle, and poultry, a sport animal or pet such as dogs, cats, horses, hamsters, rodents, or a zoo animal such as lions, tigers or bears. In one specific, non-limiting example, the non-human animal is a transgenic animal, such as, but not limited to, a transgenic mouse, cow, sheep, or goat. In one specific, non-limiting example, the transgenic animal is a mouse. In a particular example, the transgenic animal has altered proliferation and/or differentiation of a cell type as compared to a non-transgenic control (wild type) animal of the same species.

A transgenic animal contains cells that bear genetic information received, directly or indirectly, by deliberate genetic manipulation at the subcellular level, such as by microinjection or infection with a recombinant virus, such that a recombinant DNA is included in the cells of the animal. This molecule can be integrated within the animal's chromosomes, or can be included as extrachromosomally replicating DNA sequences, such as might be engineered into yeast artificial chromosomes. A transgenic animal can be a “germ cell line” transgenic animal, such that the genetic information has been taken up and incorporated into a germ line cell, therefore conferring the ability to transfer the information to offspring. If such offspring in fact possess some or all of that information, then they, too, are transgenic animals.

Transgenic animals can readily be produced by one of skill in the art. For example, transgenic animals can be produced by introducing into single cell embryos DNA encoding a marker, in a manner such that the polynucleotides are stably integrated into the DNA of germ line cells of the mature animal and inherited in normal Mendelian fashion. Advances in technologies for embryo micromanipulation permit introduction of heterologous DNA into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal.

In one non-limiting method, developing embryos are infected with a retrovirus containing the desired DNA, and a transgenic animal is produced from the infected embryo.

In another specific, non-limiting example, the appropriate DNA (s) are injected into the pronucleus or cytoplasm of embryos, preferably at the single cell stage, and the embryos are allowed to develop into mature transgenic animals.

These techniques are well known. For instance, reviews of standard laboratory procedures for microinjection of heterologous DNAs into mammalian (mouse, pig, rabbit, sheep, goat, cow) fertilized ova include Hogan et al., Manipulating the Mouse Embryo, Cold Spring Harbor Press, 1986 and Kraemer et al., Genetic Manipulation of the Early Mammalian Embryo, Cold Spring Harbor Laboratory Press, 1985.

Cells transformed so has to inactivate the expression or to inhibit the activity of the polypeptide according to any aspect of the invention, may be prepared. These cells may be useful as negative control cells in a method for screening agents that affect cell proliferation. The method may comprise contacting, administering or injecting agent candidates that may affect cell proliferation with cells, and observing or determining a reduction of cell proliferation. The method further comprises treating the negative control in the same way and further comparing the obtained results with those obtained using the negative control. The cell transformed (the negative control) may also be cultured and subsequently transplanted or grafted to a animal host. This animal host may be suitable as negative control in a screening method for candidate agent for controlling cell proliferation. Such recipient animals, together with transgenic animals lacking genes encoding the polypeptides according to the invention, or having a reduced production of a polypeptide according to the invention, or producing a polypeptide according to the invention in an inactivated, or reduced form, may be used for the screening of agents or drugs that affect cell proliferation. The transfected genes may engineered to under or over express the polypeptides of the invention and can be thus used as negative controls in these screening procedures.

For the present invention, transgenic animals under-expressing the polypeptides of the invention may be used as negative controls in screening procedures. This may be done by inducing cancerous cell growth or tumors in non-transgenic animals and transgenic animals through the use of suitable mutagens, administering a test compound to the non-transgenic animal, and comparing the results between control animals. These control animals may be transgenic animals expressing the polypeptides of the invention and untreated non-transgenic animals. Such procedures may similarly be carried out in non-transgenic recipient animals with transgenic tissue grafts.

Antibodies: The polypeptides of the invention or a fragment or conservative variants thereof can be used to produce antibodies which are immunoreactive or bind to an epitope of a polypeptide of the invention. Polyclonal antibodies, antibodies which consist essentially of pooled monoclonal antibodies with different epitopic specificities, as well as distinct monoclonal antibody preparations are included. In particular, the present invention relates to antibody(ies) that specifically binds the polypeptide according to any aspect of the invention. In particular, antibodies that specifically bind to polypeptides comprising or consisting of amino acid sequences at least 85% homologous to SEQ ID NO: 2 or a fragment thereof or to SEQ ID NO: 4 or a fragment thereof. The antibody may be selected from the group consisting of a monoclonal antibody and a polyclonal antibody. According to a particular aspect, the invention provides monoclonal and polyclonal antibodies that specifically bind to a polynucleotide according to any aspect of the invention or to a fragment thereof and do not bind to nucleostamin. Polyclonal antibodies that bind to nucleostamin are known. These are the antibodies having catalogue numbers AB5689, AB5723, and AB5691, sold by Chemicon International (a Division of Serological Corporation). According to a more particular aspect, the invention provides antibodies that specifically bind a polynucleotide according to any aspect of the invention or to a fragment thereof, wherein these antibodies are not the polyclonal antibodies: rabbit anti-human AB5689, chicken anti-human AB5723, and rabbit anti-mouse AB5691.

The preparation of polyclonal antibodies is well known to those skilled in the art. See, for example, Green et al, “Production of Polyclonal Antisera” in: Immunochemical Protocols, pages 1-5, Manson, (ed.), Humana Press, 1992; Coligan et al., “Production of Polyclonal Antisera in Rabbits, Rats, Mice and Hamsters,” in: Current Protocols in Immunology, Section 2.4.1, 1992.

The preparation of monoclonal antibodies likewise is conventional. See, for example, Coligan et al., Sections 2.5.1-2.6.7 (above); and Harlow et al in: Antibodies: a Laboratory Manual, page 726, Cold Spring Harbor Pub, 1988.

For example, monoclonal antibodies can be obtained by injecting animal, for example rabbits or mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, eg, Coligan et al, Sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, eg, syngeneic mice, to cause growth of antibody-producing tumors.

Optionally, the animals are primed with a hydrocarbon, especially oils such as pristane (tetramethylpentadecane) prior to injection. After one to three weeks, the desired monoclonal antibody is recovered from the body fluids of the animal. Antibodies can also be derived from a subhuman primate antibody. General techniques for raising therapeutically useful antibodies in baboons can be found, for example, in WO 91/11465, 1991.

Alternatively, an antibody that specifically binds a polypeptide of the invention can be derived from a humanized monoclonal antibody. Humanized monoclonal antibodies are produced by transferring mouse complementarity-determining regions from heavy and light variable chains of the mouse immunoglobulin into a human variable domain, and then substituting human residues in the framework regions of the murine counterparts.

The use of antibody components derived from humanized monoclonal antibodies obviates potential problems associated with the immunogenicity of murine constant regions. Antibodies can be derived from human antibody fragments isolated from a combinatorial immunoglobulin library. See, for example, Barbas et al, in: Methods: a CoMpanion to Methods in Enzymology, Vol. 2, page 119, 1991. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas.

Antibodies include intact molecules as well as fragments thereof, such as Fab, F (ab′) 2, and Fv which are capable of binding the epitopic determinant. These antibody fragments retain some ability to selectively bind with their antigen or receptor and are defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′) 2, the fragment of the antibody that can be obtained by treating whole antibody with the enzyme pepsin without subsequent reduction; F (ab′) 2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F (ab′) 2. This fragment can be further cleaved using a thiol reducing agent, and optionally a blocking group for the sulfhydryl groups resulting from cleavage of disulfide linkages, to produce 3. 5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Edelman et al., Methods in Enzymology, Vol. 1, page 422, Academic Press, 1967; and Coligan et al (above). at Sections 2.8.-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of VH and VL chains. This association may be noncovalent.

Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; U.S. Pat. No. 4,946,778).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of the invention as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly-used carriers which are chemically coupled to the peptide include keyhole limpet hemocyanin (KLH), thyroglobulin, bovine serum albumin (BSA), and tetanus toxoid. The coupled peptide is then used to immunize the animal (eg, a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal antibody.

Binding affinity for a target antigen is typically measured or determined by standard antibody-antigen assays, such as competitive assays, saturation assays, or immunoassays such as enzyme-linked immunosorbent assay (ELISA) or radioimmuno assay (RIA). Such assays can be used to determine the dissociation constant of the antibody. The phrase “dissociation constant” refers to the affinity of an antibody for an antigen. Specificity of binding between an antibody and an antigen exists if the dissociation constant (KD=1/K, where K is the affinity constant) of the antibody is, for example <1 llM, 100 nM, or <0.1 nM.

Antibody molecules will typically have a KD in the lower ranges. KD=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, eg, therapeutic, diagnostic, or detection moieties can be linked to an antibody that specifically binds a polypeptide of the invention, using any number of means known to those of skill in the art. Exemplary effector molecules include, but not limited to, radiolabels, fluorescent markers, or toxins (eg Pseudomonas exotoxin (PE), see U.S. Pat. No. 4,545,985 and U.S. Pat. No. 4,894,443, for a discussion of toxins and conjugation). Both covalent and noncovalent attachment means may be used.

The procedure for attaching an effector molecule to an antibody varies according to the chemical structure of the effector. Polypeptides typically contain a variety of functional groups; eg, carboxylic acid (COOH), free amine (—NH2) or sulfhydryl (—SH) groups, which are available for reaction with a suitable functional group on an antibody to result in the binding of the effector molecule. Alternatively, the antibody is derivatized to expose or attach additional reactive functional groups.

The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule.

Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (eg, through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (eg, when exposed to tumor-associated enzymes or acidic pH) may be used.

In view of the large number of methods that have been reported for attaching a variety of radiodiagnostic compounds, radiotherapeutic compounds, label (eg enzymes or fluorescent molecules) drugs, toxins, and other agents to antibodies, one skilled in the art will be able to determine a suitable method for attaching a given agent to an antibody or other polypeptide.

From the teachings above, on the production of antibodies against the polypeptides of the invention, it will be apparent that a kit comprising such antibodies may be produced for the diagnosis or treatment of a condition associated with a polypeptide of the invention. Such a kit can comprise packaging, information pertaining to the antibody and/or polypeptide of the invention, containers and storage media or buffer, chemicals and other components that facilitate the use of the antibodies in a clinical or laboratory setting.

Methods of inducing differentiation and/or inhibiting proliferation: A method for inhibiting proliferation of a cell is disclosed herein. This method encompasses altering the level of a polypeptide of the invention in the cell by various means, thereby inhibiting proliferation of the cell of the cell. The cell can be in vivo or in vitro.

Expression of a polypeptide of the invention can be either increased or decreased to induce differentiation and/or inhibit proliferation. In one example, expression of a polypeptide of the invention is increased as compared to a control. Increased expression includes, but is not limited to, at least a 20% increase in the amount of mRNA coding for a polypeptide of the invention or a polypeptide of the invention in a cell as compared to a control, such as, but not limited to, at least a 30%, 50%, 75%, 100%, or 200% increase of the mRNA or polypeptide.

In another example, expression of a polypeptide of the invention is decreased as compared to a control. Decreased expression includes, but is not limited to, at least a 20% decrease in the amount of mRNA or polypeptide in a cell as compared to a control, such as, but not limited to, at least a 30%, 50%, 75%, 100%, or 200% decrease of RNA or polypeptide in the cell. Suitable controls include a cell not contacted with an agent that alters expression of a polypeptide of the invention, such as a wild-type cell, a stem cell, or an untreated tumor cell. Suitable controls also include standard values.

In a further aspect, a GNL3L polypeptide is a conservative variant of SEQ ID NO: 2, such that it includes no more than fifty conservative amino acid substitutions, such as no more than two, no more than five, no more than ten, no more than twenty, or no more than fifty conservative amino acid substitutions in SEQ ID NO: 2. In another aspect, a GNL3L polypeptide has an amino acid sequence as set forth as SEQ ID NO: 2.

Specific, non-limiting examples of a GNL3L polypeptide or Grn1p polypeptide of use in the methods disclosed herein is a conservative variant of SEQ ID NO: 2 or a conservative variant of SEQ ID NO: 4, as described above. In several aspects, a conservative variant includes at most one, at most two, at most five, at most ten, or at most fifteen conservative substitutions of the sequence shown in SEQ ID NO: 2 or SEQ ID NO: 4. Generally, a conservative variant will bind to antibodies that immunoreact with a polypeptide including a sequence set forth as SEQ ID NO: 2, and/or will immunoreact with a polypeptide including a sequence set forth as SEQ ID NO: 4.

In the methods disclosed herein, prevalence or expression of a polypeptide of the invention can either be increased or decreased in a cell to inhibit proliferation of the cell or to induce differentiation of the cell. In one aspect, a polypeptide of the invention is administered to the cell of interest. In another aspect, the activity of a polypeptide of the invention is inhibited. In another aspect, expression of a nucleic acid encoding a polypeptide of the invention is induced. In a further aspect, expression of a nucleic acid encoding a polypeptide of the invention is decreased.

Differentiation can be induced, or proliferation decreased, of any cell, either in vivo or in vitro, using the methods disclosed herein. In one aspect, the cell is a stem cell, such as, but not limited to, an embryonic stem cell, a neuronal progenitor cell, a hematopoietic stem cell, or a pancreatic endocrine progenitor cell.

In one aspect, the cell is a tumor cell, including a cell of a benign or a malignant tumor (eg a cancer cell). Cancer cells include, but are not limited to, tumors of the breast, intestine, liver, lung, ovary, testes, bone, lymphocytes, bladder, skin, prostate, brain, kidney, endocrine system, thyroid, or any other tissue or organ of interest.

In yet another aspect, expression of a polypeptide of the invention is increased or decreased in a sarcoma, eg an osteosarcoma or Kaposi's sarcoma. In a specific, non-limiting example, a nucleic acid encoding a polypeptide of the invention is provided in a viral vector and delivered by way of a viral particle which has been derivatized with antibodies immunoselective for an osteosarcoma cell (see, for example, U.S. Pat. No. 4,564,517 and U.S. Pat. No. 4,444,744).

In a still further aspect, expression of a polypeptide of the invention is altered (increased or decreased) in tissue which is characterized by unwanted de-differentiation and which may also be undergoing unwanted apoptosis. For instance, many neurological disorders are associated with degeneration of discrete populations of neuronal elements. For example, Alzheimer's disease is associated with deficits in several neurotransmitter systems, both those that project to the neocortex and those that reside with the cortex.

Altering the expression or activity of a polypeptide of the invention can also be used to inhibit proliferation of smooth muscle cells, and can therefore be used as part of a therapeutic regimen in the treatment of a patient suffering from a condition which is characterized by excessive smooth muscle proliferation. The arterial wall is a complex multicellular structure and is important in the regulation of inflammation, coagulation, and regional blood flow. Vascular smooth muscle cells (SMCs) are located predominantly in the arterial tunica media and are important regulators of vascular tone and blood pressure. These cells are normally maintained in a nonproliferative state in vivo. Arterial injury results in the migration of SMCs into the intimal layer of the arterial wall, where they proliferate and synthesize extracellular matrix components.

Arterial intimal thickening after injury is the result of the following series of events: (1) initiation of smooth muscle cell proliferation within hours of injury, (2) SMC migration to the intima, and (3) further SMC proliferation in the intima with deposition of matrix. The overall disease process can be termed a hyperproliferative vascular disease because of the etiology of the disease process.

This process can be biologically induced (as in atherosclerosis, transplant atheroscelerosis) or mechanically induced (as in balloon angioplasty). Thus, a method is provided herein of altering smooth muscle cell proliferation by altering the expression of the polypeptide of the invention.

The level of a polypeptide of the invention in a cell can be altered by administration of a polypeptide of the invention. For example, a polypeptide of the invention can be administered using liposomes, or any other method known to be effective in delivering proteins known to one of skill in the art.

Expression of a polypeptide of the invention can be altered by administering a nucleic acid encoding the polypeptide to the cell. In vitro methods for delivery of a nucleic acid are disclosed above. In vivo, expression constructs including a nucleic acid encoding a polypeptide of the invention can be administered in any biologically effective carrier, eg any formulation or composition capable of effectively transfecting cells in vivo.

Approaches include insertion of a nucleic acid encoding a polypeptide of the invention in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), poly-lysine conjugates, gramacidin S, artificial viral envelopes or other such intracellular carriers, as well as direct injection of the gene construct or CaPO₄ precipitation carried out in vivo. The particular delivery system of use will depend on such factors as the phenotype of the intended target and the route of administration, e.g. locally or systemically.

In one aspect, a viral vector containing nucleic acid, eg, a cDNA, encoding a polypeptide of the invention is utilized. These vectors include, but are not limited, to retroviruses or adenoviruses. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses, and defective retroviruses are well characterized for use in gene transfer. Thus, recombinant retrovirus can be constructed in which part of the retroviral coding sequence (gag, pol, env) has been replaced by nucleic acid encoding a polypeptide of the invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F M et al. (eds.), Greene Publishing Associates, Sections 9.10-9. 14, 1989. Exemplary retroviruses include pLJ, PZIP, pWE and pEM, which are of use in transfecting neural cells, epithelial cells, endothelial cells, lymphocytes, myoblasts, hepatocytes, bone marrow cells, in vitro and/or in vivo (see for example U.S. Pat. No. 6,460,6464; U.S. Pat. No. 4,868,116; U.S. Pat. No. 4,980,286; WO 89/07136; WO 89/02468; WO 89/05345; and WO 92/07573).

It has been shown that it is possible to limit the infection spectrum of retroviruses and consequently of retroviral-based vectors, by modifying the viral packaging proteins on the surface of the viral particle (see, for example WO 93/25234, WO 94/06920, and WO 94/11524). For instance, strategies for the modification of the infection spectrum of retroviral vectors include coupling antibodies specific for cell surface antigens to the viral env protein. Coupling can be in the form of the chemical cross-linking with a protein or other variety (eg lactose to convert the env protein to an asialoglycoprotein), as well as by generating fusion proteins (eg single-chain antibody/env fusion proteins). Retroviral gene delivery can be further enhanced by the use of tissue- or cell-specific transcriptional regulatory sequences which control expression of the CCR-gene of the retroviral vector.

Adenovirus-derived vectors are also of use with a nucleic acid encoding a polypeptide of the invention. The genome of an adenovirus can be manipulated such that it encodes a gene product of the invention, but is inactivate in terms of its ability to replicate in a normal lytic viral life cycle. Suitable adenoviral vectors are derived from the adenovirus strain Ad type 5 dl324 or other strains of adenovirus (eg, Ad2, Ad3, Ad7, etc). The adenovirus can be a replication-defective adenoviral vector, such as a virus deleted for all or parts of the viral E1 and E3 genes (see, Graham et al. in Methods in Molecular Biology, E. J. Murray, (ed.; Humana, Clifton, N.J., vol. 7. pp. 109-127, 1991).

Yet another viral vector system of use is the adeno-associated virus (AAV). Adeno-associated virus is a naturally occurring defective virus that requires another virus, such as an adenovirus or a herpes virus, as a helper virus for efficient replication and a productive life cycle. Other viral vector systems that are of use include herpes virus, vaccinia virus, and other RNA viruses, such as lentiviruses.

In addition to viral transfer methods, non-viral methods can also be employed. Exemplary delivery systems of this type include liposomal derived systems, poly-lysine conjugates, and artificial viral envelopes. In one specific, non-limiting example, a nucleic acid encoding a polypeptide of the invention can be delivered to a cell of interest using liposomes bearing positive charges on their surface (eg, lipofectins). These liposomes can be tagged with antibodies against cell surface antigens of the target tissue (eg see WO 91/06309; Japanese Patent Application 1047381; and European Patent Publication EP-A-43075). In another specific, non-limiting example, the delivery system includes an antibody or cell surface ligand which is cross-linked with a nucleic acid binding agent such as poly-lysine (see, for example, WO 93/04701, WO 92/22635, WO 92/20316, WO 92/19749 and WO 92/06180).

Expression of a polypeptide of the invention may be altered by administering an antisense molecule or a ribozyme that specifically binds the polypeptide, or by administering antisense, ribozymes or small inhibitory RNA molecules (siRNA). Antisense molecules are oligonucleotide probes or their derivatives which specifically hybridize (eg bind) under cellular conditions, with the cellular mRNA and/or genomic DNA encoding the polypeptide, so as to inhibit or interfere with the expression of that protein, eg by inhibiting transcription and/or translation. The binding may be by conventional base pair complementarity, or, for example, in the case of binding to DNA duplexes, through specific interactions in the major groove of the double helix.

Antisense nucleic acids, namely DNA or RNA molecules that are complementary to at least a portion of the nucleic acid sequence encoding for a polypeptide of the invention can be used in the methods disclosed herein. In one specific example, in the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are of use, since they are easily synthesized and are less likely to cause problems than larger molecules when introduced into the target cell producing a polypeptide of the invention. The use of antisense methods to inhibit the in vitro translation of genes is well known in the art.

Use of an oligonucleotide to stall transcription is known as the triplex strategy since the oligomer winds around double-helical DNA, forming a three-strand helix. Therefore, these triplex compounds can be designed to recognize a unique site on a chosen gene. This strategy can be used to produce oligonucleotides that specifically inhibit transcription of RNA encoding a polypeptide of the invention.

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it. A major advantage of this approach is that, because they are sequence-specific, only mRNAs with particular sequences are inactivated.

There are two basic types of ribozymes namely, tetraxymena-type and “hammerhead”-type. Tetrahymena-type ribozymes recognize sequences which are four bases in length, while hammerhead-type ribozymes recognize base sequences 11-18 bases in length. The longer the recognition sequence, the greater the likelihood that the sequence will occur exclusively in the target mRNA species. Either type of ribozyme is of use in inhibiting expression of a polypeptide of the invention.

The present disclosure further provides a method for treating mammalian cells by interfering or inhibiting expression of a polypeptide of the invention in the cells, by exposing the animal cells to an effective amount of an RNA (siRNA) suitable for interfering or inhibiting expression of a polypeptide of the invention. The RNA comprises double stranded RNA of about 15 to about 40 nucleotides containing a 0-nucleotide to 5-nucleotide long overhang on the 3′ and/or 5′ strands, wherein the sequence of the RNA is substantially identical to a portion of a mRNA or transcript of a polypeptide of the invention.

The siRNA can be used to inhibit a polypeptide of the invention suitable, either in vivo and in vitro. The inhibitory RNAs can have unmodified or modified backbones and/or component nucleosides. Such modifications include, but are not limited to, thio, 2′-fluro 2′-amino, 2′-doxy, 4-thio, 5-bromo, 5-iodo and 5-(3-aminoallyl) derivatives of ribonucleosides. The siRNA can be delivered directly, derived from a viral RNA, or produced from a transgene.

An antisense or small inhibitory RNA construct can be delivered, for example, as an expression plasmid containing elements such as promoters and enhancers necessary for the expression of the siRNA, which, when transcribed in the cell, produces RNA which is complementary to at least a unique portion of the cellular mRNA. Such expression plasmids may be delivered by viral vectors as taught by US 2005/0106731.

Alternatively, the antisense or siRNA construct is an oligonucleotide probe which is generated ex vivo and which, when introduced into the cell causes inhibition of expression by hybridizing with the mRNA and/or genomic sequences encoding one of the subject's CCR proteins. Such oligonucleotide probes are preferably modified oligonucleotide which are resistant to endogenous nucleases, eg exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. No. 5,176,996; U.S. Pat. No. 5,264,564; and U.S. Pat. No. 5,256,775).

Pharmaceutical preparations and therapy: In one aspect, a method is provided for inhibiting or decreasing proliferation of a cell in a subject, including administering a therapeutically effective amount of an agent that alters the level of a polypeptide of the invention, and a pharmaceutically acceptable carrier. A polypeptide of the invention may be a polypeptide including an amino acid sequence at least 85% identical to SEQ ID NO: 2 or SEQ ID NO: 4. Administering the pharmaceutical composition can be accomplished by any means known to one of skill in the art.

The present invention also provide a composition, for example a pharmaceutical composition or medicament, comprising at least one of the polypeptide according to the invention. For example, the invention provides a composition, for example a pharmaceutical composition or medicament, comprising a polypeptide comprising or consisting of an amino acid sequence at least 85%, 90%, 95%, 98%, 99% or 100% homologous to the amino acid sequence selected from the group consisting of SEQ ID NO: 2 and SEQ ID NO: 4.

The pharmaceutical compositions are preferably prepared and administered in dose units. Solid dose units are tablets, capsules and suppositories. For treatment of a subject, such as but not limited to a human subject, and depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administrations of subdivided doses at specific intervals.

The pharmaceutical compositions can be administered systemically or locally, such as, but not limited to, by injection directly into a tumor. The compositions are in general administered topically, intravenously, intramuscularly, orally, parenterally, or as implants, but even rectal use is possible in principle.

Suitable solid or liquid pharmaceutical preparation forms are, for example, granules, powders, tablets, coated tablets, (micro) capsules, suppositories, syrups, emulsions, suspensions, creams, aerosols, drops or injectable solutions in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

A therapeutically effective dose of an agent that alters the level of a polypeptide of the invention is the quantity of a compound necessary to inhibit, to cure or at least partially arrest the symptoms of the disorder and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, eg, in Gilman et al, (eds.), Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

In clinical settings, systems for the introduction of a nucleic acid encoding a polypeptide of the invention, or a polynucleotide designed to inhibit the expression of a polypeptide of the invention, can be introduced into a subject by any of a number of methods. For instance, a pharmaceutical preparation of the nucleic acid delivery system can be introduced systemically, e.g. by intravenous injection, and specific transduction of the target cells occurs predominantly from specificity of transfection provided by the gene delivery vehicle, the cell-type or tissue-type expression due to the transcriptional regulatory sequences controlling expression of the gene, or a combination thereof.

In other aspects, initial delivery of the recombinant gene is more limited with introduction into the animal being quite localized. For example, the gene delivery vehicle can be introduced by catheter (see U.S. Pat. No. 5,328,470) or by stereotactic injection.

Moreover, the pharmaceutical preparation can consist essentially of the nucleic acid system in an acceptable diluent, or can be a slow release matrix in which the nucleic acid delivery vehicle is imbedded. Alternatively, where the complete delivery system can be produced from recombinant cells, e.g. retroviral packages, the pharmaceutical preparation can include one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinacious biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of viral particles by cells implanted at a particular target site. Such aspects can be used for the delivery of an exogenously purified virus, which has been incorporated in the polymeric device, or for the delivery of viral particles produced by a cell encapsulated in the polymeric device. By choice of monomer composition or polymerization technique, the amount of water, porosity and consequent permeability characteristics can be controlled.

The selection of the shape, size, polymer, and method for implantation can be determined on an individual basis according to the disorder to be treated and the individual patient response. The generation of such implants is generally known in the art (see, for example, Concise Encyclopedia of Medical & Dental Materials, ed. by David Williams (MIT Press: Cambridge, Mass., 1990; U.S. Pat. No. 4,883,666). In another aspect of an implant, a source of cells producing a recombinant virus is encapsulated in implantable hollow fibers. Such fibers can be pre-spun and subsequently loaded with the viral source (U.S. Pat. No. 4,892,538; U.S. Pat. No. 5,106,627), or can be co-extruded with a polymer which acts to form a polymeric coat about the viral packaging cells (U.S. Pat. No. 4,391,909; U.S. Pat. No. 4,353,888). Again, manipulation of the polymer can be carried out to provide for optimal release of viral particles.

Thus, a kit can be provided containing an agent that affects that proliferation of a cell and with other elements of a delivery system as described above. The kit may further comprise instructions for use such that the kit may be readily employed in a clinical setting.

Screening for agents that affect proliferation of a cell: A method for screening for agents that affect cell proliferation is provided herein. Agents of interest such as antibodies can bind to the polypeptides of the invention. Thus, in one aspect, the method to identify an agent of interest includes contacting candidate agents with polypeptides comprising or consisting of an amino acid sequence at least 85%, 90%, 95%, 98%, 99%, or 100% identical to the polypeptides of the invention with an agent of interest in vitro. This binding then evaluated. A decrease in the binding of the agent with a polypeptide of the invention indicates that the agent may affect the proliferation of the cell since the polypeptides of the invention have been shown to have such an effect. Suitable controls include the binding of the agent and the polynucleotides of the invention in the absence of any agent or in the presence of a carrier, such as a buffer. A suitable control also includes the first agent and a polynucleotide of invention in the presence of an another compound or agent known to affect this interaction. Suitable controls also include standard values. “Incubating” includes conditions which allow contact between the test agent or compound and the agent and/or the polynucleotides of the invention. “Contacting” includes such reactions in solution and/or solid phase.

Prior to performing any assays to detect interference with the association of a test agent with the polynucleotides of the invention, rapid screening assays could be used to screen a large number of candidate agents to determine if they bind to the first agent or the polynucleotide of the invention. Rapid screening assays for detecting binding to HIV proteins have been disclosed, for example, in U.S. Pat. No. 5,230,998. In this type of assay, the first agent or the polynucleotide of the invention is incubated with a first antibody capable of binding to the first agent or the polynucleotides of the invention, and the candidate agent to be screened. Excess unbound first antibody is washed and removed, and antibody bound to the first agent or polynucleotides of the invention is detected by adding a second labeled antibody which binds the first antibody. Excess unbound second antibody is then removed, and the amount of the label is quantitated. The effect of the binding effect is then determined as a percentage by the formula: (quantity of the label in the absence of the drug)−(quantity of the label in the presence of the drug/quantity of the label in the absence of the drug)×100. Agents that are found to have a high binding affinity to the first agent or polynucleotide of the invention may then be used in other assays more specifically designed to test inhibition of the interaction.

Examples of agents that interfere with an interaction of an agent and a polynucleotide of the invention, identified using such an assay, include: chemical compounds; fragments and fusions of polynucleotide of the invention; peptidomimetics; antibodies; synthetic ligands that bind polynucleotide of the invention or its agent, other agents which cause the disassociation of the agent and polynucleotide of the invention; appropriate fragments of the polynucleotide of the invention or its agent, or other fragments of the natural or synthetic ligands or chemical compounds which bind to agent and prevent the interaction of the agent and the polynucleotide of the invention, and thereby affect cell proliferation and/or other cellular activities.

The test compound may also be a combinatorial library for screening a plurality of compounds. Compounds identified in the disclosed methods can be further evaluated, detected, cloned, sequenced, and the like, either in solution of after binding to a solid support, by any method usually applied to the detection of a specific DNA sequence, such as PCR, oligomer restriction, allele-specific oligonucleotide (ASO) probe analysis, oligonucleotide ligation assays (OLAs), and the like.

Binding can be measured by any means known to one of skill in the art. For example competitive binding assays can be utilized. In another example, a polypeptide, such as a polypeptide comprising or consisting of an amino acid sequence at least 85% identical to SEQ ID NO: 2 or SEQ ID NO: 4, is attached to a matrix, or introduced into wells of a microtiter plate. Extracts that contain normal or modified forms of the agent are incubated with the matrices or plates, and the agent adsorbs onto the polynucleotide of the invention but not onto control matrices or wells that lack the polynucleotide of the invention. After washing away the unabsorbed agent, the matrices or plates are analyzed by standard methods such as ELISA for detection of the adsorbed agent.

Drug candidates are added to the assay wells to determine whether any agent, such as a chemical compound, antibody or peptide, blocks binding of an agent to the matrices or plates that contain the polynucleotide of the invention. The assays could also be done inversely, by binding an agent and by studying the adsorption of polynucleotide of the invention onto the agent. Such assays can also be performed with small fragments of an agent that contain only the domain needed for binding to the polynucleotide of the invention.

In the present application, we describe two new nucleolar GTPases from the same GTPase subfamily (FIG. 10), Grn1p from the fission yeast and FLJ10613 (GNL3L) from human. GNL3L has so far been described only as a ‘hypothetical’ protein. The present inventors, for the first time, found that FLJ10613 (GNL3L) is a protein, isolated the protein and determined its function.

The nucleolus is the principal site for the generation of rRNA as also for ribosome assembly and maturation. Many aspects of rRNA processing, ribosome biogenesis and its nuclear export of ribosomes are conserved between yeast and humans (see Tschochner and Hurt, 2003; Venema and Tollervey, 1999 and references therein). The initial rRNA processing is concomitant with the formation of the 90S ribosomal precursor particle that separates into 40S and 60S pre-subunits. The pre-60S ribosomes undergo a series of rRNA processing reactions that begin within the nucleolus followed by export of the ribosomes through the nuclear pore complex (NPC) (Fatica and Tollervey, 2002; Milkereit et al., 2001; Nissan et al., 2002; Tschochner and Hurt, 2003; Venema and Tollervey, 1999). Nucleotide-binding proteins comprising several putative GTPases are known to be associated with pre-60S ribosomes on their journey from the nucleolus to the cytoplasm (Nissan et al., 2002; Tschochner and Hurt, 2003). However, their precise function at the molecular level is unclear.

Here, the present inventors report the open reading frame (ORF) of one of these sequences, SPBC26H8.08c, encodes a new gene Grn1 (GTPase in Ribosomal export from the Nucleolus) expressing a GTPase. The present inventors also identified the protein, human FLJ10613 (GNL3L), as a homolog of Grn1p since its expression complements the growth defect in a grn1 null mutant. Furthermore, there is also provided evidence that GNL3L is required for growth of human cells. Further, there are also provided methods that utilize this information in the control or inhibition of cell proliferation.

The present inventors employed the fission yeast as a model system to understand the role of these GTPases in cell growth. Mature rRNA species were reduced markedly in a grn1Δ (null mutant) with a concomitant accumulation of 35S pre-rRNA transcript. Using a GFP-reporter assay, the inventors demonstrate that grn1Δ fails to export the ribosomal protein Rpl25a from the nucleolus into the cytoplasm. Deleting any of the Grn1p G-domain motifs resulted in a null phenotype and nuclear/nucleolar localization consistent with the lack of nucleolar export of pre-ribosomes. Heterologous expression of GNL3L in a grn1 restores processing of 35S pre-rRNA and nuclear export of Rpl25a. Genetic complementation in yeast and SiRNA knockdown in HeLa cells confirmed the homologous proteins Grn1p and GNL3L are required for growth. The inventors proved here that GTpases Grn1p and GNL3L are active in coupled events involving processing of pre-rRNA and export of pre-ribosomes from the nucleolus.

Having now generally described the invention, the same will be more readily understood through reference to the following examples which are provided by way of illustration, and are not intended to be limiting of the present invention.

### EXAMPLES

### Example 1

### Materials and Methods

TABLE I
S. pombe yeast strains used

| | Parent | | | |
|---|---|---|---|---|
| Strains (YNB) | strain | Plasmid | Marker | Reference |
| YNB544 | YNB484 | pBNB190 | Leu2 | This example |
| YNB545 | YNB484 | pBNB189 | Leu2 | This example |
| YNB546 | YNB484 | pCDL280 | Leu2 | This example |
| YNB566 | YNB484 | pBNB203 | Leu2 | This example |
| YNB567 | YNB484 | pBNB202 | Leu2 | This example |
| YNB568 | YNB484 | pBNB204 | Leu2 | This example |
| YNB611 | YNB484 | pBNB217 | Leu2 | This example |
| YNB631 | YNB484 | pBNB221 | Leu2 | This example |
| YNB795 | YNB484 | pBNB284 | Leu2 | This example |
| YNB805 | YNB484 | pBNB316 | Leu2 | This example |
| YNB858 | | leu1-32 ura4-D18 his3-Δ1, | G418 | This example |
| | | ΔSPBC26H8.08c::GNL3L-FLAG | | |
| | | KanMX6 h- | | |
| YNB859 | | leu1-32 ura4-D18 his3-Δ1, | G418 | This example |
| | | SPBC26H8.08c::FLAG | | |
| | | KanMX6 h- | | |
| YNB860 | YNB484 | pBNB395 | Leu2 | This example |
| YNB956 | YNB484 | pBNB412 | Leu2 | This example |
| YNB1075 | YNB858 | pBNB221 | G418, | This example |
| | | | Leu2 | |
| YNB1076 | YNB859 | pBNB221 | G418, | This example |
| | | | Leu2 | |
| YNB951 | YNB484 | pBNB475 | Leu2 | This example |
| YNB952 | YNB484 | pBNB476 | Leu2 | This example |
| YNB953 | YNB484 | pBNB477 | Leu2 | This example |

Yeast strains were maintained in YES (Yeast Extract plus Supplements) or EMM (Edinburgh Minimal Media) medium supplemented with appropriate amino acids and +/−15 μM thiamine routinely to repress or induce respectively the nmt1 promoter (Moreno et al., 1991). Unless otherwise specified, yeast cultures were maintained or grown at 320C and harvested at 0.4-1.0 OD600 for all experiments. YNB483 leu1-32 ura4-D18 his3-D1 and YNB484 leu1-32 ura4-D18 DSPBC26H8.08c::ura4+ his3-D1 were the principal yeast strains used in this study and are referred to in the text as either wild type and null mutant or grn1Δ respectively. PCR-based gene integration of GNL3L into the Grn1 locus using the kanMX6 marker was performed using previously described procedures (Bahler et al., 1998; Chen et al., 2004). Plasmids generated for this study are described in Table 2.

TABLE 2
Plasmids/constructs

| Construct | Description | Marker | Reference |
|---|---|---|---|
| pBNB168 | pBlueScript KS II-Ura4 | Amp | Chen et al |
| pBNB189 | ΔG1 (AA²⁷⁶⁻²⁸³): Nucleotides encoding | Amp | This example |
| | AA^{1-275 and} AA²⁸⁴⁻⁴⁷⁰ were generated by | | |
| | PCR using pBNB190 as template with | | |
| | NB380 + NB379 and NB378 + NB377 sets | | |
| | of respective primers. The two fragments | | |
| | were used in fusion PCR using primers | | |
| | NB380 and NB377. The PCR product | | |
| | was inserted into pCDL280 as in | | |
| | pBNB190. | | |
| pBNB190 | The full-length Grn1 gene without its | Amp | This example |
| | intron was amplified by PCR using S. pombe | | |
| | genomic DNA as template and | | |
| | primers NB380 and NB377. The resulting | | |
| | Grn1 was digested with SalI and NotI and | | |
| | inserted immediately upstream of the | | |
| | GFP gene in vector pCDL280. | | |
| pBNB202 | ΔCC (AA⁷⁰⁻⁹⁰): Nucleotides encoding | Amp | This example |
| | AA¹⁻⁶⁹ and AA⁹¹⁻⁴⁷⁰ were generated by | | |
| | PCR using BNB190 as template with | | |
| | NB380 + NB454 and NB453 + NB377 sets | | |
| | of respective primers. The resulting | | |
| | fragments were used in fusion PCR with | | |
| | NB380 and NB377 as the primers. The | | |
| | PCR product was inserted into pCDL280 | | |
| | as in pBNB190. | | |
| pBNB203 | ΔG5 (AA¹⁶⁴⁻¹¹⁵): Nucleotides | Amp | This example |
| | corresponding to AA¹⁻¹⁶³ and AA¹⁷⁶⁻⁴⁷⁰ | | |
| | were amplified by PCR using BNB190 as | | |
| | template with NB380 + NB458 and | | |
| | NB457 + NB377 sets of respective | | |
| | primers. The products were used for | | |
| | fusion PCR with primers NB380 and | | |
| | NB377. The resulting fragment was | | |
| | inserted in pCDL280 as in pBNB190. | | |
| pBNB204 | ΔRG (AA⁴⁰⁵⁻⁴¹⁵): Nucleotides | Amp | This example |
| | corresponding to AA¹⁻⁴⁰⁴ and AA⁴¹⁶⁻⁴⁷⁰ | | |
| | were amplified by PCR using BNB190 as | | |
| | template with NB380 + NB456 and | | |
| | NB455 + NB377 sets of respective | | |
| | primers. The PCR products were used for | | |
| | fusion PCR with primers NB380 and | | |
| | NB377. The resulting product was | | |
| | inserted in pCDL280 as in pBNB190. | | |
| pBNB217 | ΔG4 (AA¹⁹⁵⁻²⁰⁸): Nucleotides | Amp | This example |
| | corresponding to AA¹⁻¹⁹⁴ and AA²⁰⁹⁻⁴⁷⁰ | | |
| | were amplified by PCR using BNB190 as | | |
| | template with NB380 + NB525 and | | |
| | NB524 + NB377 sets of respective | | |
| | primers. The PCR products were used for | | |
| | fusion PCR with primers NB380 and | | |
| | NB377. The resulting product was | | |
| | inserted in pCDL280 as in pBNB190. | | |
| pBNB221 | The RpI25a gene (SPBC106.18) was | Amp | This example |
| | generated without its intron. The N- and | | |
| | C-terminal fragments were amplified by | | |
| | PCR using the genomic DNA as template | | |
| | with NB535 + NB541 and NB540 + NB536 | | |
| | sets of respective primers. The fusion | | |
| | product was obtained by PCR using N- | | |
| | and C-terminal products as template with | | |
| | NB535 and NB536 as primers. The | | |
| | resulting product was inserted into | | |
| | pCDL280 using SalI and NotI. | | |
| pBNB284 | The full-length human NGP1 gene was | Amp | This example |
| | amplified by PCR using a HeLa cDNA | | |
| | library as template, with primers NB712 | | |
| | and NB713. The PCR products were | | |
| | digested with SalI and NotI and inserted | | |
| | in pCDL280 similarly as for pBNB190. | | |
| pBNB316 | The full-length GNL3L gene was | Amp | This example |
| | amplified by PCR using a HeLa cDNA | | |
| | library as template and primers NB762 | | |
| | and NB763. The PCR product was | | |
| | cloned into pCDL280 immediately | | |
| | upstream of GFP gene with SalI and | | |
| | NotI. | | |
| pCDNA3.1 | | Amp | Novagen |
| pBNB335 | ΔG5-GFP fusion was amplified using | Amp | This example |
| | pBNB203 as template and | | |
| | NB864 + NB865 as primers. The PCR was | | |
| | cloned into pCDNA3.1 with Kpn I and | | |
| | Xho I. | | |
| pBNB336 | ΔG4-GFP fusion gene was cloned into | Amp | This example |
| | pCDNA3.1 following the same strategy as | | |
| | in pBNB335 except using pBNB217 as | | |
| | template for PCR. | | |
| pBNB337 | ΔG1-GFP fusion gene was cloned in | Amp | This example |
| | pCDNA3.1 following the same strategy as | | |
| | in pBNB335 using pBNB189 as template. | | |
| pBNB338 | The Grn1-GFP fusion gene was cloned | Amp | This example |
| | into pCDNA3.1 following the same | | |
| | strategy as in pBNB335 using pBNB190. | | |
| pBNB339 | ΔRG-GFP fusion gene was cloned into | Amp | This example |
| | pCDNA3.1 following the same strategy as | | |
| | in pBNB335 using pBNB204. | | |
| pBNB340 | GFP was released from pBNB8 by | Amp | This example |
| | BamHI/XhoI and inserted in pCDNA3.1 | | |
| | containing the same unique sites. | | |
| pBNB341 | GNL3L-GFP fusion gene was cloned into | Amp | This example |
| | pCDNA3.1 following the same strategy as | | |
| | pBNB335 cloning using pBNB316 as | | |
| | template and NB883 + NB865 as primers. | | |
| pBNB343 | pBlueScript KS II-KanMX6 | Amp | Chen et al |
| pBNB373 | Nucleotides encoding the FLAG epitope | Amp | This example |
| | were annealed and cloned into pBNB341 | | |
| | with NotI and XhoI. FLAG replaces GFP | | |
| | in this vector. | | |
| pBNB376 | Primers NB907 + NB908 were annealed to | Kan | This example |
| | form a siRNA fragment specific for | | |
| | GNL3L at nt1047-1065 and inserted in | | |
| | the pSIREN shuttle vector (BD | | |
| | Biosciences). | | |
| pBNB377 | Primers NB909 + NB910 were annealed to | Kan | This example |
| | form a scrambled version of GNL3L | | |
| | siRNA (pBNB376) and inserted in | | |
| | pSIREN shuttle vector. | | |
| pBNB378 | Oligonucleotides specific for the | Kan | This example |
| | Luciferase gene were annealed to form a | | |
| | siRNA fragment and inserted in pSIREN | | |
| | shuttle vector. | | |
| pBNB395 | Nucleotides encoding the FLAG epitope | Amp | This example |
| | were annealed and cloned into pBNB316 | | |
| | with NotI and XhoI. FLAG replaces GFP | | |
| | in this vector. | | |
| pBNB396 | The KanMX6 cassette (BNB343) was | Amp | This example |
| | inserted into pBNB373 using ApaI and | | |
| | XbaI. | | |
| pBNB412 | ΔG3: Nucleotides encoding AA¹⁻³²⁵ and | Amp | This example |
| | AA³³⁰⁻⁴⁷⁰ were generated by PCR using | | |
| | BNB190 as template with | | |
| | NB380 + NB1007 and NB1006 + NB377 | | |
| | sets of respective primers. The two | | |
| | fragments were used in fusion PCR using | | |
| | primers NB380 and NB377. The resulting | | |
| | product was inserted in pCDL280 as in | | |
| | pBNB190. | | |
| pBNB475 | To clone the Grn1 ORF together with its | Amp | This example |
| | native promoter (398 nts upstream of | | |
| | gene ORF arbitrarily determined to be the | | |
| | promoter), primers NB1149 (promoter | | |
| | sequence) and NB1150 (Grn1 N-terminal | | |
| | sequence containing an inherent BamHI | | |
| | site) were used to PCR-amplify a | | |
| | fragment comprising promoter and ˜360 nts | | |
| | of Grn1 N-terminal sequence | | |
| | using yeast genomic DNA as the | | |
| | template. The fragment was digested with | | |
| | SalI & BamHI and cloned into pBNB190. | | |
| | The fragment thus replaced the ˜360 nts | | |
| | of original Grn1 N-terminal sequence in | | |
| | the new plasmid. Subsequently, | | |
| | promoter-Grn1-GFP was released from | | |
| | the above vector using SacI and BgIII, | | |
| | and cloned into pHL1288. | | |
| pBNB476 | To clone the Grn1 ΔNLS1 mutant (AA₆₋₂₂) | Amp | This example |
| | under its native promoter, a fusion PCR | | |
| | strategy was employed. 5′ and 3′ end | | |
| | PCR products were amplified by using | | |
| | respective sets of primers | | |
| | NB1149 + NB1152 (Grn1 5′end sequence | | |
| | with removal of NLS1) and NB1151 | | |
| | (complementary to NB1152) + NB1150, | | |
| | and pBNB475 as the template. The | | |
| | fusion was obtained by using 5′ and 3′ | | |
| | end PCR products as templates and | | |
| | NB1149 + NB1150 as primers. The | | |
| | resulting product was digested with SacI | | |
| | & BamHI and cloned into pBNB475 | | |
| | treated with the same pair of enzymes. | | |
| | The fragment promoter-Grn1 ΔNLS1 | | |
| | replaced the original promoter-Grn1 N- | | |
| | terminal sequence (˜360 nts). | | |
| pBNB477 | To clone the Grn1 ΔNLS2 mutant (AA₆₋₃₆) | Amp | This example |
| | under its native promoter, the cloning | | |
| | strategy was similar to that of pBNB476. | | |
| | 5′ and 3′ end PCR products were | | |
| | amplified by using respective sets of | | |
| | primers NB1149 + NB1154 (Grn1 5′end | | |
| | sequence with removal of NLS2) and | | |
| | NB1153 (complementary to NB1152) + NB1150, | | |
| | using pBNB475 as template. | | |
| | The fusion PCR was achieved by using | | |
| | the above two PCR products as | | |
| | templates and NB1149 + NB1150 as | | |
| | primers. The fragment was digested with | | |
| | SacI & BamHI and cloned into pBNB475 | | |
| | treated with the same pair of enzymes. | | |
| | The promoter-Grn1 ΔNLS2 replaced the | | |
| | original sequence. | | |

Plasmid constructions. DNA fragments used to create plasmids for this example were generated by PCR using high fidelity enzyme Turbo Pfu (Stratagene). Oligonucleotide primers are listed in Table 3. All constructs were confirmed by DNA sequencing.

TABLE 3
Primers (^{F} = Forward; ^{R} = Reverse)

| | |
|---|---|
| NB110^{F} | GGTTAAAAAAGAATAATCGGTAATGTTTTTTCTCTAGACAAC |
| | CAACTGTAAAATTTGTAACTACAGCATTTTTTACAATGCAAC |
| | AGCTATGACCGGCTACCATTCACCCGCTCAACCCTCACTAAA |
| | GGGAAC |
| | (SEQ ID NO:5) |
| NB111^{R} | GAAAAACCGCAACCGAAAACCAAATCCCAAAATATAAGCTCT |
| | AAGCAACAATAGCTTTTTTCGTAAGTTGAAAACTCTCATTGT |
| | AAAACGACGGCCCGTTCTGCCGAGCATGACGACACTATAGGG |
| | CGAATTGG |
| | (SEQ ID NO:6) |
| NB373⁷ | GCATTGCTAAACTAAGGAAATCTTTCTAAATGTGAATATAAA |
| | TTACTAATTAGCTTCAACTTTAAAAATAACGAGGGAATTCGA |
| | GCTCGTTTAAAC |
| | (SEQ ID NO:7) |
| NB375^{R} | GTTCTTCTTTTACTCTTTTTTTCTTAAGAAATAAGTTAGAAA |
| | TAGTTACGCGTGCATATACTTACTTAAGGAAACTTTGTATAG |
| | TTCATCC |
| | (SEQ ID NO:8) |
| NB377^{R} | CATCTGCGGCCGCGGAAAATCATTAAGGTCAAA |
| | (SEQ ID NO:9) |
| NB378^{F} | CTTACAGTCGGTGTAATTTCTGTTATTAACGCTCTT |
| | (SEQ ID NO:10) |
| NB379^{R} | AAGAGCGTTAATAACAGAAATTACACCGACTGTAAG |
| | (SEQ ID NO:11) |
| NB380^{F} | CATATGTCGACTATGGTTTCCTTAAAAAAAAAGAGTAAAAGA |
| | AG |
| | (SEQ ID NO:12) |
| NB453^{F} | GATCGAAGAACAGAAGCGCGAAGACGCTGTTGATGAA |
| | (SEQ ID NO:13) |
| NB454^{R} | TTCATCAACAGCGTCTTCGCGCTTCTGTTCTTCGATC |
| | (SEQ ID NO:14) |
| NB455^{F} | GCTACTGATTTTTTAGTCAATATTATTCCAAATCTTAACGCT |
| | GC |
| | (SEQ ID NO:15) |
| NB456^{R} | GCAGCGTTAAGATTTGGAATAATATTGACTAAAAAATCAGTA |
| | GC |
| | (SEQ ID NO:16) |
| NB457^{F} | AAGTTGTTGAAGCGTCAGAAGGGACTCGTTCAAAAG |
| | (SEQ ID NO:17) |
| NB458^{R} | CTTTTGAACGAGTCCCTTCTGACGCTTCAACAACTT |
| | (SEQ ID NO:18) |
| NB524^{F} | GCATCTTCTGCTGAAGAATCAGAAGTACTCAACAAG |
| | (SEQ ID NO:19) |
| NB525^{R} | CTTGTTGAGTACTTCTGATTCTTCAGCAGAAGATGC |
| | (SEQ ID NO:20) |
| NB629^{R} | CCCAAAAAGTTAAAAGATGG |
| | (SEQ ID NO:21) |
| NB631^{R} | TCGTTCAACACCTCATC |
| | (SEQ ID NO:22) |
| NB700^{R} | TCGTTAGAGGTGAGACAA |
| | (SEQ ID NO:23) |
| NB702^{F} | AGAAGTGGAAAAGGAGAC |
| | (SEQ ID NO:24) |
| NB712^{F} | CATATGTCGACTATGGTGAAGCCCAAGTACAAAGG |
| | (SEQ ID NO:25) |
| NB713^{R} | CATCTGCGGCCGCGGCTGCTTTTGTCTGAATTTT |
| | (SEQ ID NO:26) |
| NB762^{F} | CATATGTCGACTATGATGAAACTTAGACACAA |
| | (SEQ ID NO:27) |
| NB763^{R} | CATCTGCGGCCGCGGGTCACCAACACCATCATCAGC |
| | (SEQ ID NO:28) |
| NB864^{F} | GACTCAGGTACCATGGTTTCCTTAAAAAAAAAG |
| | (SEQ ID NO:29) |
| NB865^{R} | GACTCACTCGAGCTATTTGTATAGTTCAT |
| | (SEQ ID NO:30) |
| NB883^{F} | GACTCAGGTACCATGATGAAACTTAGACACAA |
| | (SEQ ID NO:31) |
| NB907^{F} | GATCCGCTATTATGGCGTCTCTGGGTTCAAGAGACCCAGAGA |
| | CGCCATAATAGCTTTTTTGGTACCG |
| | (SEQ ID NO:32) |
| NB908^{R} | AATTCGGTACCAAAAAAGCTATTATGGCGTCTCTGGGTCTCT |
| | TGAACCCAGAGACGCCATAATAGCG |
| | (SEQ ID NO:33) |
| NB909^{F} | GATCCGCTATATTGCGGTCTGGTCGTTCAAGAGACGACCAGA |
| | CCGCAATATAGCTTTTTTGGTACCG |
| | (SEQ ID NO:34) |
| NB910^{R} | AATTCGGTACCAAAAAAGCTATATTGCGGTCTGGTCGTCTCT |
| | TGAACGACCAGACCGCAATATAGCG |
| | (SEQ ID NO:35) |
| NB963^{F} | GGCCGCAGATGGACTACAAGGATGACGATGACAAATAAC |
| | (SEQ ID NO:36) |
| NB964^{R} | TCGAGTTATTTGTCATCGTCATCCTTGTAGTCCATCTGC |
| | (SEQ ID NO:37) |
| NB1002^{F} | GTTAAAAAAGAATAATCGGTAATGTTTTTTCTCTAGACAACC |
| | AACTGTAAAATTTGTAACTACAGCATTTTTTACAATGATGAA |
| | ACTTAGACACAA |
| | (SEQ ID NO:38) |
| NB1004^{F} | CGAAAAGAACTCATCTGAAGTTCAGGATACTCAAATCGTTAC |
| | TGAGTGGGCCAAAGAATTTGACCTTAATGATTTTCCGCGGCC |
| | GCAGATGGACTAC |
| | (SEQ ID NO:39) |
| NB1006^{F} | AACAAATTACGTTTGGTCATTGTTTTTCCTTCTAGT |
| | (SEQ ID NO:40) |
| NB1007^{R} | ACTAGAAGGAAAAACAATGACCAAACGTAATTTGTT |
| | (SEQ ID NO:41) |
| NB1102^{F} | GATATAATTAATTCAGAC |
| | (SEQ ID NO:42) |
| NB1125^{R} | AACCGCAACCGAAAACCAAATCCCAAAATATAAGCTCTAAGC |
| | AACAATAGCTTTTTTCGTAAGTTGAAAACTCTCTAGAACTAG |
| | TGGATCTG |
| | (SEQ ID NO:43) |
| NB1149^{F} | GACGCAGGTACCGTCGACGAGCTCCTTTATATTAAAAATTAT |
| | TAATTGC |
| | (SEQ ID NO:44) |
| NB1150^{R} | GCTATCTTTGAAATCATTAGGGATCCAT |
| | (SEQ ID NO:45) |
| NB1151^{F} | ACTACAGCATTTTTTACAATGGTTTCCTTAAAAGCTGC |
| | (SEQ ID NO:46) |
| NB1152^{R} | GCAGCTTTTAAGGAAACCATTGTAAAAAATGCTGTAGT |
| | (SEQ ID NO:47) |
| NB1153^{F} | ACTACAGCATTTTTTACAATGGTTTCCTTAAAAAATCCGC |
| | (SEQ ID NO:48) |
| NB1154^{R} | GCGGATTTTTTAAGGAAACCATTGTAAAAAATGCTGTAGT |
| | (SEQ ID NO:49) |
| NB1160^{F} | CATATGTCGACTATGAAAAGGCCTAAGTTAAAG |
| | (SEQ ID NO:50) |
| NB1161^{R} | CATCTGCGGCCGCGGCACATAATCTGTACTGAAGTC |
| | (SEQ ID NO:51) |
| NB1478^{F} | TTTCGCTGCGTTCTTC |
| | (SEQ ID NO:52) |

Deletion of Grn1. The Ura4-marker cassette with SPBC26H8.08c flanking (5′ and 3′) homology regions was generated by PCR using the primers NB110 and NB111. This 2.2 Kb fragment was directly used to transform the homozygous diploid YNB400 (ade6-M210/ade6-M216 leu1-32 ura4-D18 his3-A1, h+/h−). Diploid transformants were selected on EMM-ura-ade plates. Sporulation of the heterozygous diploid and dissection of tetrads from at least 24 independent asci yielded four haploid spores/tetrad. On germination, two of the four spores from each tetrad grew extremely slowly on rich medium. Each one of these slow-growing colonies was confirmed as having the SPBC26H8.08c deletion by colony PCR using 5′ and 3′primers flanking the gene.

Construction of strains YNB858 and YNB859 by gene integration. Oligonucleotides representing the FLAG sequence (AGATGGACTACAAGGATGACGATGACAAATAA) (SEQ ID NO:53) were annealed and inserted into pBNB341 to replace the GFP ORF to generate pBNB373. Next, the gene encoding KanMX6 (BNB343) was inserted into pBNB373 downstream of GNL3L-FLAG fusion in the reverse orientation (pBNB396). The GNL3L-FLAG-KanMX6 cassette was amplified by PCR using pBNB396 as the template with the primers NB1004 (5′ end)+NB1125 (3′ end). FLAG-KanMX6 cassette was amplified using the same template and 3′ end primer as for the GNL3L cassette while 5′ end primer (NB1002) corresponded to 76 nts of Grn1 ORF sequence immediately upstream of its stop codon. The integrations resulted in the replacement of Grn1 ORF with GNL3L-FLAG-KanMX6 (YNB858) and the fusion of Grn1 with FLAG epitope (YNB859).

Fluorescence microscopy. Fission yeast cells were prepared for DAPI, GFP fluorescence or Indirect-immunofluorescence as previously described (Balasundaram et al., 1999; Chen et al., 2004; Varadarajan et al., 2005). For some figures as noted, the GFP and DAPI images of a single nucleus were enlarged and digitally manipulated to convert one color to another in order to render a sharper contrast and thus render and delineate more vividly the nucleolar region from the extra-nucleolar region.

All epi-fluorescence microscopy were performed at 1000× magnification using a Leica DMLB microscope equipped with an Optronics DEI-750T coded CCD camera with Leica Qwin proprietary software. For some experiments, samples were viewed with an upright Nikon E800 confocal microscope and images were acquired using a Nikon DXM1200 camera with Image Pro-plus 4.5 software (Media Cybernetics). Adobe Photoshop 5.0 was used for all image presentations.

Cos-7 cells in chamber culture slides (BD Biosciences) were infected with vaccinia virus vTF7-3 and transfected with GNL3L-GFP (BNB341) and Grn1-GFP (BNB338) expression plasmids using Lipofectin (Invitrogen). After 12 h, cells were fixed with 3% paraformaldehyde and mounted in mounting medium (Vector laboratories). Localization of GNL3L and Grn1 was determined by confocal microscopy. Nucleoli were revealed by immunostaining with anti-Nucleolin.

Biochemical methods. Standard laboratory techniques were employed for extraction of DNA, total RNA or protein and to perform southern (DNA), northern (RNA), or western (protein) blots.

siRNA knockdown. A unique sequence of GNL3L (nt1047-1065) was chosen as the target sequence for RNA interference. NB907-908 containing the target sequence, CTATTATGGCGTCTCTGGG (SEQ ID NO:54) and a scrambled version, CTATATTGCGGTCTGGTCG (SEQ ID NO:55) (used as a negative control) were cloned into the PSIREN vector (BD Biosciences) under the control of the human U6 promotor. A siRNA targeted to the Luciferase gene was used as an additional control. All siRNA expressing constructs (9 μg of each) were co-transfected with pcDNA3 vector (0.9 μg) into HeLa cells. After 120 hr selection in G418 (500 μg/ml), the cells were photographed and total RNA were isolated using spin minicolumns according to manufacturers instructions. Reverse transcription-Polymerase Chain Reaction (RT-PCR) was performed by a standard protocol and GNL3L-specific signal was amplified using the following primers:

GNL3L Forward:

5′ATGTGCGAATTCATGATGAAACTTAGACACAAAAATAAAAAGCC3′

(SEQ ID NO:56) and GNL3L Reverse:

(SEQ ID NO:57)

5′CACCATGATATCCCGGATGAACTTGTCCAGGTAGAC3′.

β-actin was amplified with primers forward: 5′GGCGACGAGGCCCAGA3′ (SEQ ID NO:58) and reverse: 5′CGATTTCCCGCTCGGC (SEQ ID NO:59) as an internal control to normalize the equal quantity of RT products were used in PCR.

Rpl25a localization for Δgrn1, Grn1-FLAG and ΔGrn1:GNL3L-FLAG strains. Strains expressing nmt1:Rpl25a:GFP were cultured in EMM medium supplemented with appropriate amino acids and 15 μM thiamine were grown to log-phase after which, the cells were washed in medium without thiamine and diluted to an OD₅₉₅ value of 0.1 in fresh medium with (nmt1 OFF) without B1 (nmt1 ON). Samples of cells were harvested at early log-phase (OD₅₉₅ 0.5-0.8), stained with DAPI and examined for DAPI and GFP fluorescence.

RNA extraction and northern analysis. Total RNA were isolated by phenol:chloroform method following standard methods and was analyzed on a 1.2% agarose-acrylamide gel. After electrophoresis, ethidium-bromide stained-RNA bands were imaged to record 25S and 18S mature rRNA species and then transferred onto Hybond™ N+ membrane (Amersham Biociences, Bucks, UK). All oligonucleotide probes were based largely on Good et al., 1997 and are shown in FIG. 3. To identify 35S pre-rRNA species, a DIG-labeled PCR probe specific for 5′ETS was synthesized using S. pombe genomic DNA as template, DIG-DNA labeling Mix as substrate and primer sets of NB700+NB702 (all commercial reagents for northern analysis were from Roche, Mannheim, Germany). The resulting PCR product corresponded to the 5′-3′sequence—900 nucleotides upstream of the 18S rRNA ORF. The 5.8S probe corresponds to a sequence within the 5.8S ORF (NB1478), whereas the ITS1 oligonucleotide probes (NB629 and NB1102) corresponded to the D→A2 and A3→B1 cleavage sites respectively and the ITS2 oligonucleotide probe (NB631) corresponding to the sequence within E→C1 cleavage sites. All four were generated by 3′end labeling using DIG-11-ddUTP according to manufacturer's instructions. Hybridization of RNA with the above probes was performed in commercially available buffers. For 5′ETS northern, hybridization was performed at 55° C. and washes at 65° C., whereas for 5.8S, ITS1 and ITS2, hybridization and washes were performed at 30° C. The membrane containing RNA was hybridized with above probes and subsequently detected with anti-DIG AP fragment and developed using CSPD according to the manufacturer's instructions. A commercial actin RNA DIG-labeled probe was used to detect yeast act1 mRNA levels as an internal control.

Western analysis. To analyze the expression of GFP- or FLAG-tagged Grn1p or GNL3L in yeast, 5-10 ml cultures were grown in appropriate medium and harvested at OD₅₉₅ values of 0.5-1.0. Proteins were extracted by resuspending cell pellets in 1% SDS-PBS buffer and lysing them with an equal volume of glass beads in a bead-beater for 4×30 secs at 4° C. In some instances, to concentrate protein, TCA was added to the above yeast lysate at 4° C. to a final concentration of 25%. Precipitated proteins were re-suspended in SDS/sample buffer containing 8 M urea and pH adjusted with 1.0M Tris-base buffer. After separation on 12% SDS-PAGE gels, proteins were transferred to PVDF membranes (Millipore, Bedford, Mass.). Specific epitope-tagged proteins were visualized by their reaction either with polyclonal anti-GFP (Molecular Probes, Eugene, Oreg.) or polyclonal anti-FLAG (Sigma, St. Louis, Mo.).

### Example 2

### Grn1p is a Member of a Novel G-Protein Family

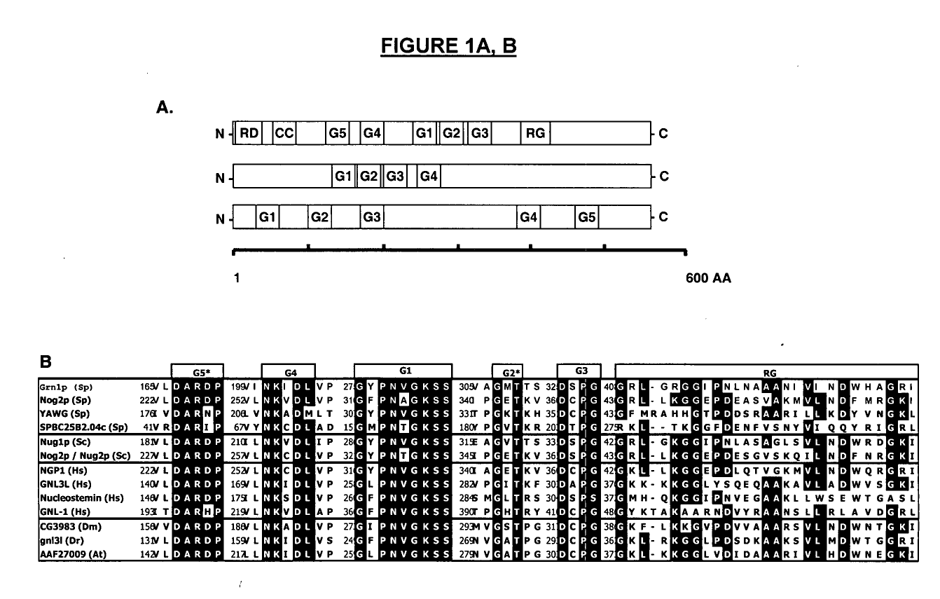

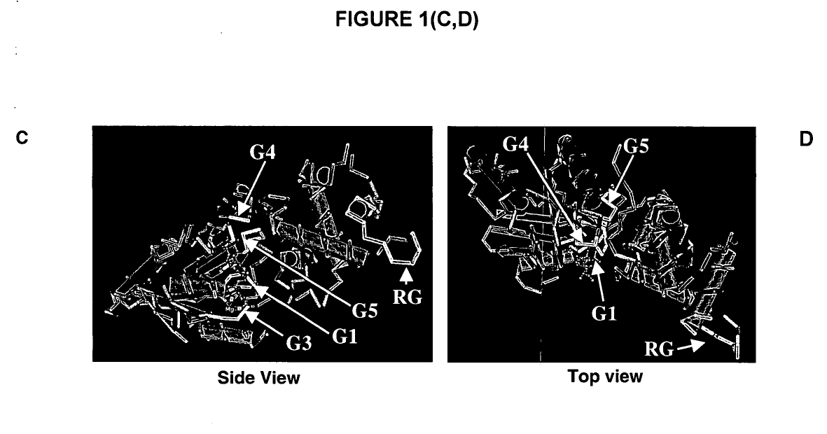

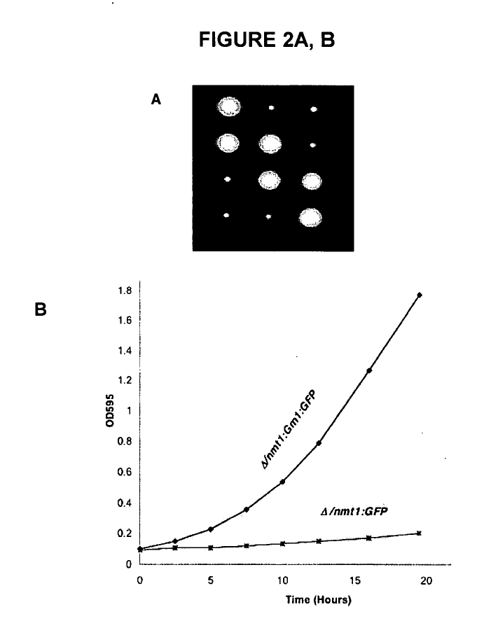

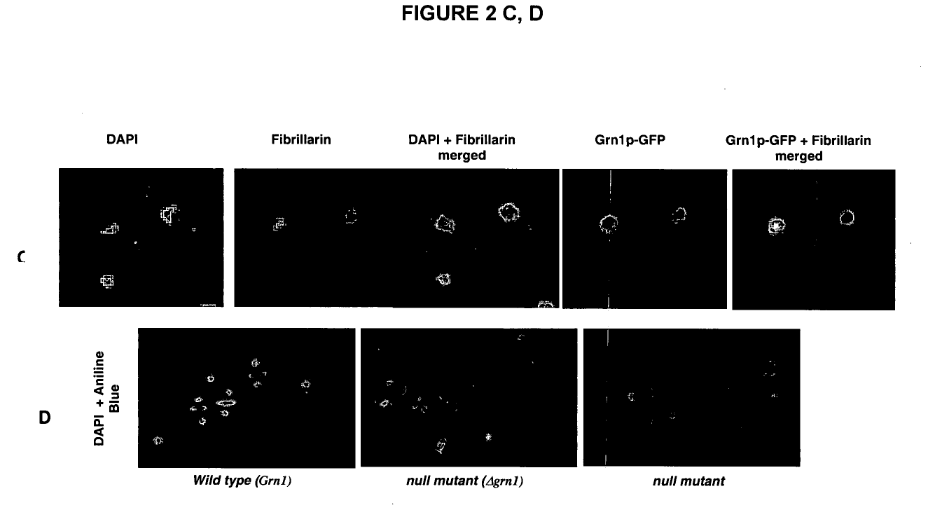

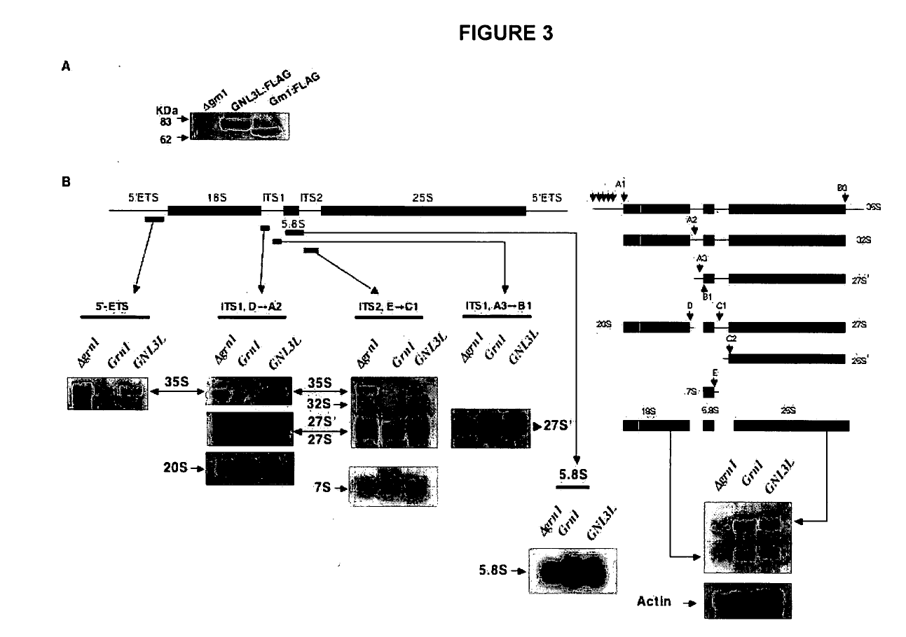

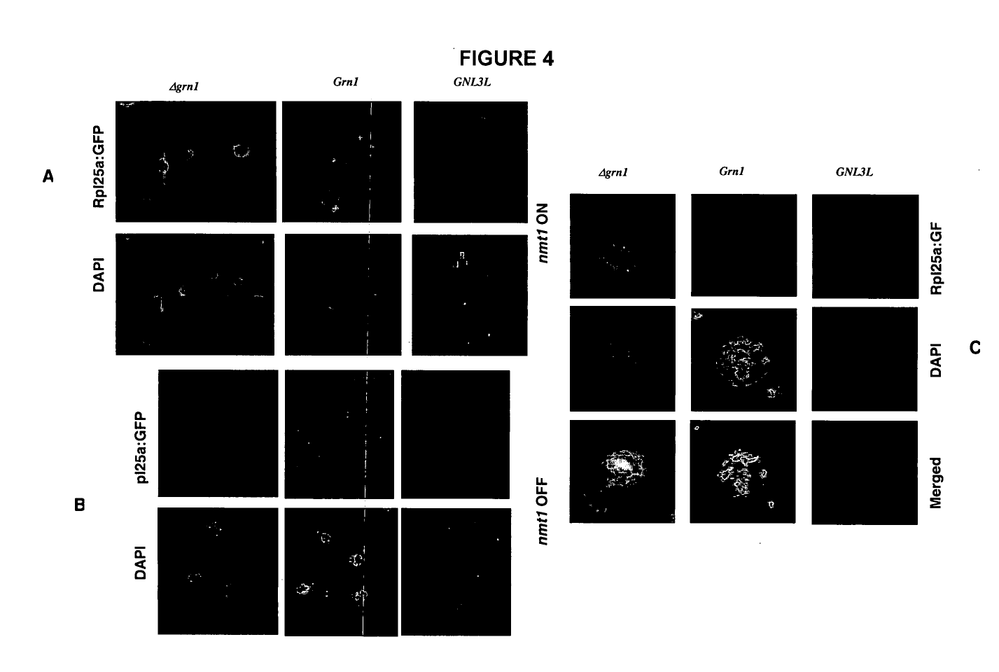

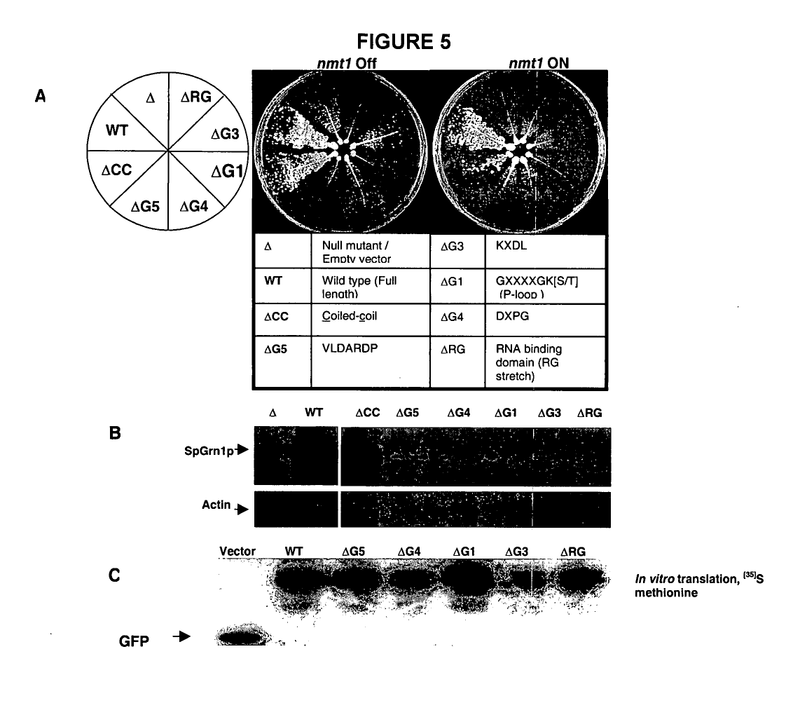

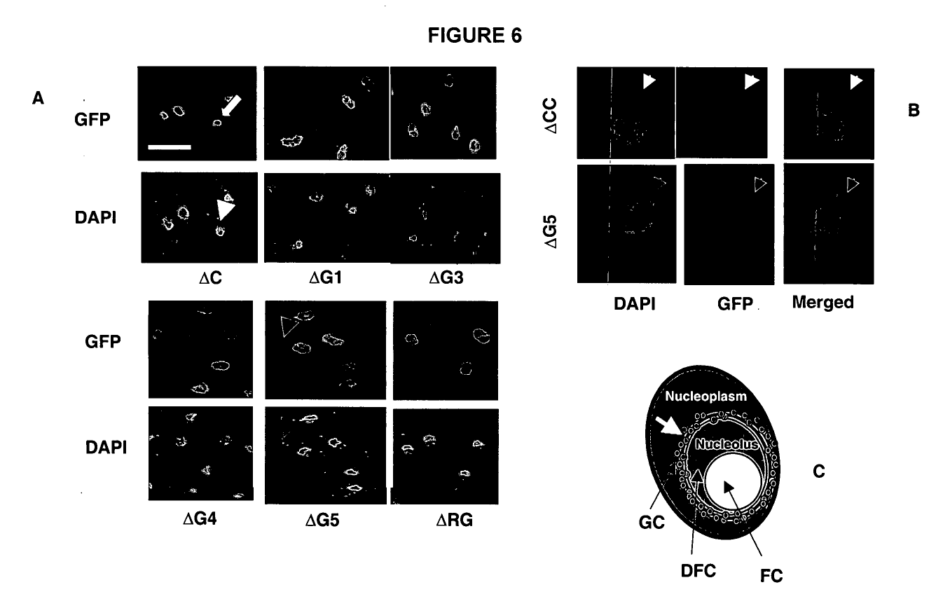

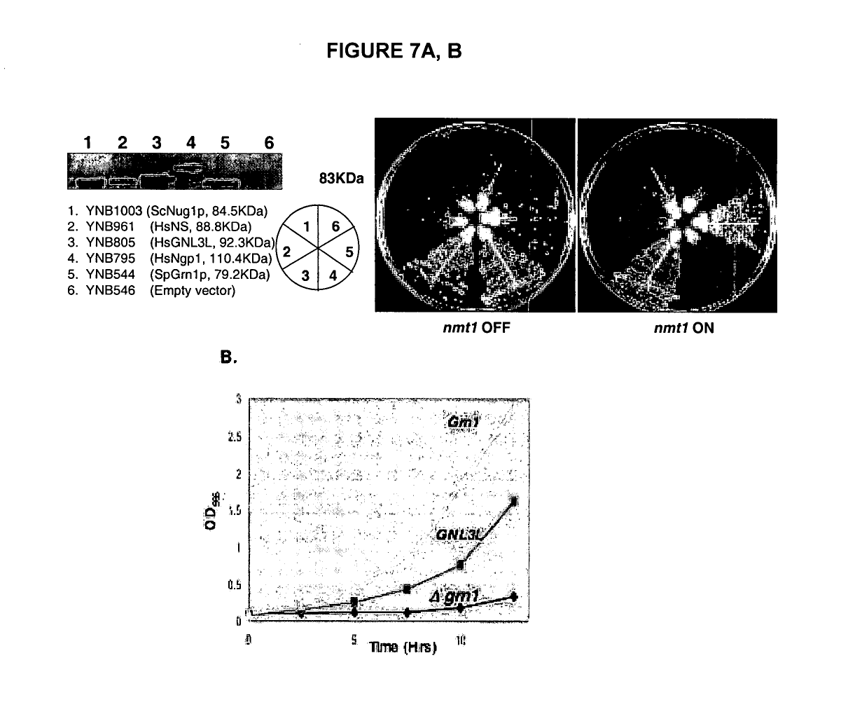

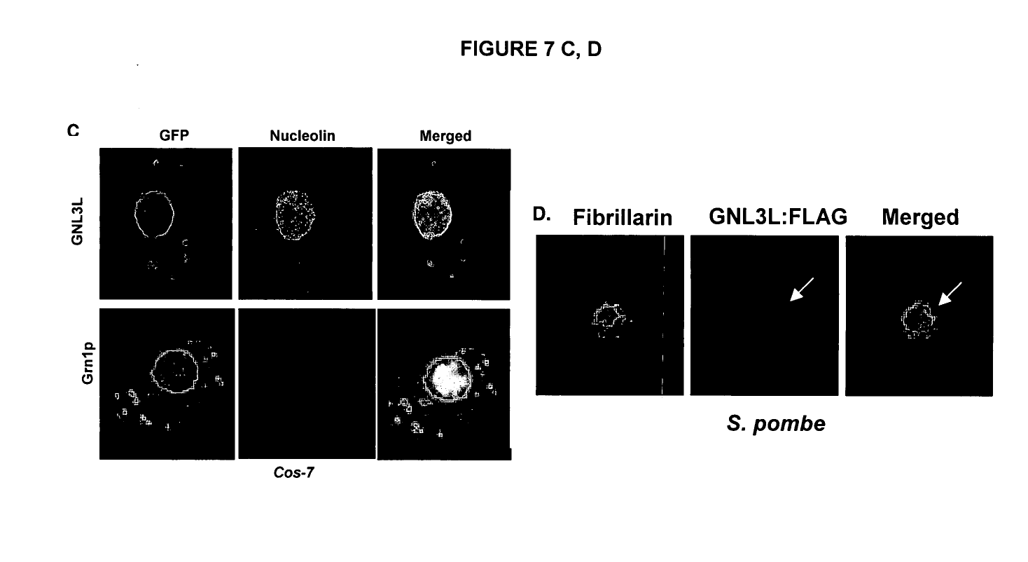

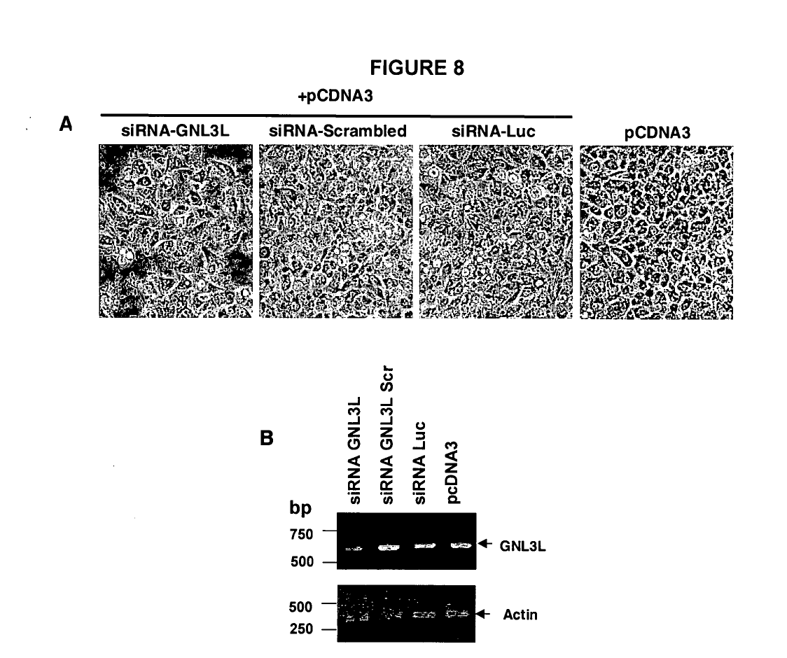

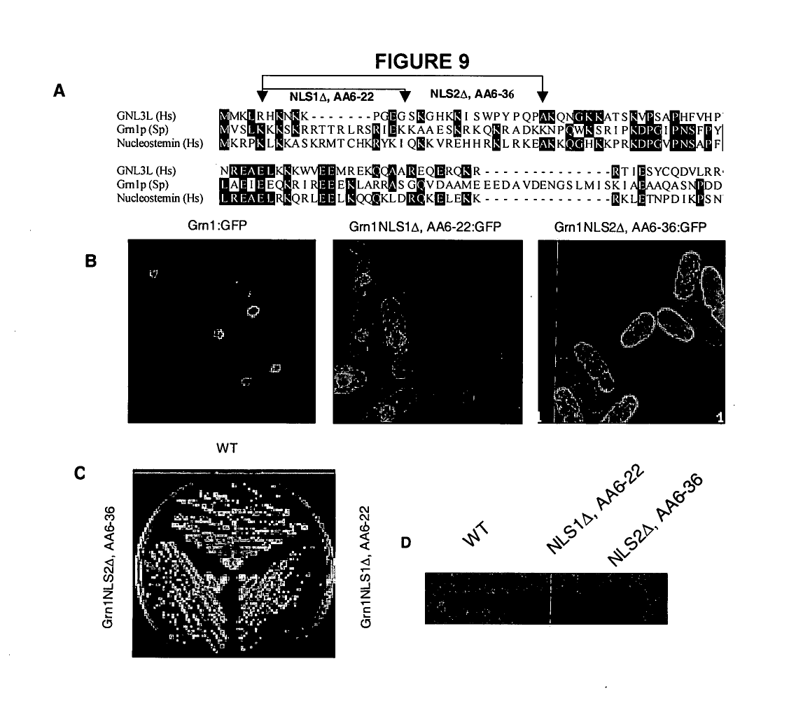

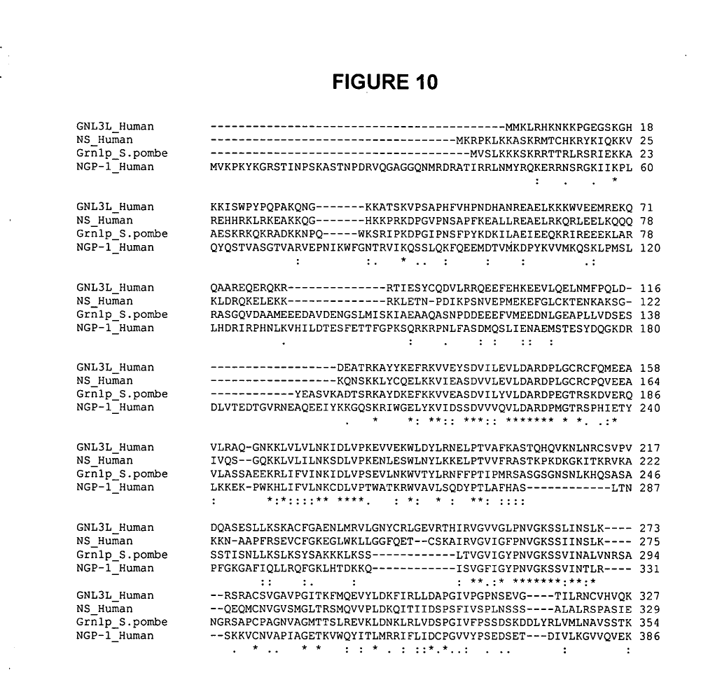

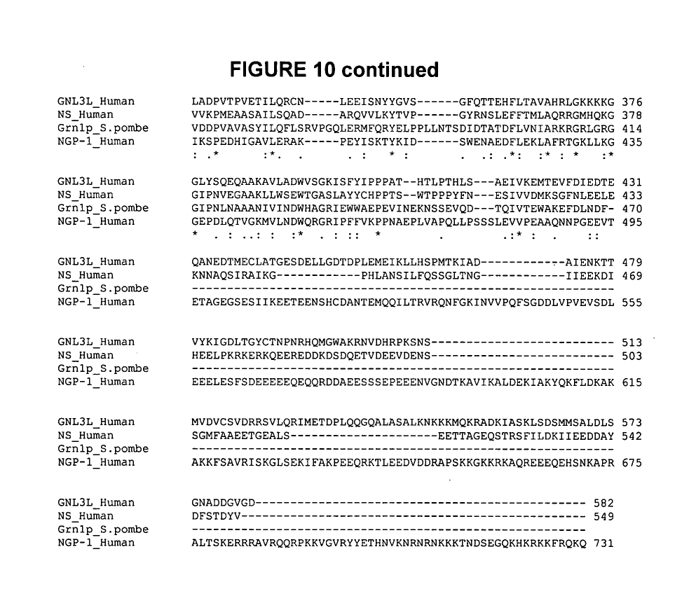

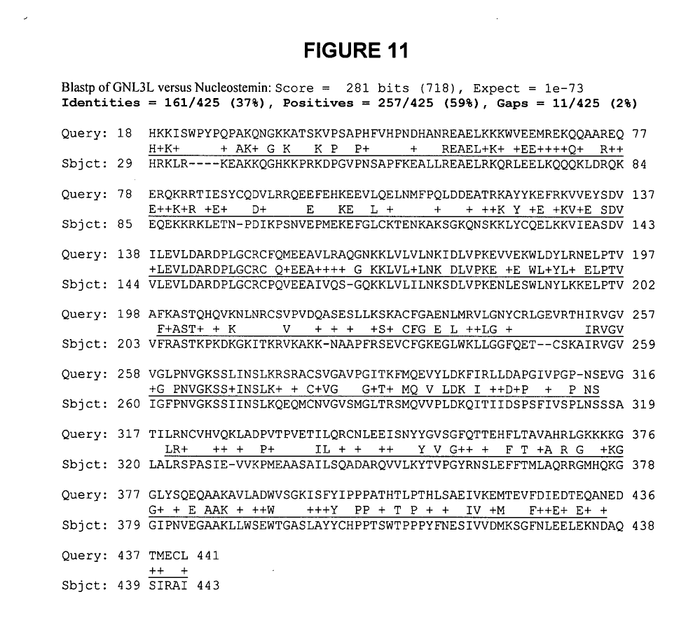

Grn1 encodes a predicted protein of 470 residues. PSORT analysis (Nakai and Horton, 1999) identifies a predicted coiled-coil domain and at least four GTPase-consensus motifs designated here as G1, G3, G4 and a G5* sequence that define a G protein (Leipe et al., 2002; Takai et al., 2001) (FIG. 1A). In addition, there is a putative RNA-binding domain at the C-terminus (RG-stretch). A BLASTp search (Altschul et al., 1997) of the predicted protein data banks showed that highly related sequences are found in yeast as well as in diverse eukaryotes with the ‘G’-domain displaying an extremely high degree of sequence homology (FIG. 1B). A CD-search of conserved domain databases (CDD) (Marchler-Bauer et al., 2005), Pfam (Bateman et al., 2004) and Clusters of Orthologous Groups (COGs) (Tatusov et al., 2000) revealed some very interesting aspects of this GTPase.

All the diagnostic motifs of the GTPases described above were present albeit in altered juxtaposition to each other. Rather than the usual G1-G2-G3-G4-G5, found in the superfamily of regulatory GTP hydrolases (Leipe et al., 2002; Takai et al., 2001), the G1 motif (GXXXXGK(S/T) or P-loop is between the G4 motif (KXDL) and G3 motif (DXXG/DXPG) as G5*-G4-G1-G2*-G3 (FIG. 1A) in what has been described as a circularly permuted G-motif (Daigle et al., 2002; Leipe et al., 2002).

Thus, the domain structure of the putative GTPase like other previously studied nucleolar GTPases, Nug1p (Bassler et al., 2001), Nog2p (Nug2p) (Saveanu et al., 2001), Ngp1, (Racevskis et al., 1996) and NS (Tsai and McKay, 2002; Tsai and McKay, 2005) conforms to that observed for the HSR1_MMR1_GTP-binding protein GNL-1, (Vernet et al., 1994) and members of the Ylqf/YawG family of GTPases (Leipe et al., 2002). The so-called G2* (YAFTT or Effector or Switch I is a less conserved motif and not present in all GTPases) and G5* (EXSAX) (Takai et al., 2001) appear to be ill-defined in this group.

However, in keeping with a previous report (Saveanu et al., 2001), the amino acid residues DARDP and GxT will to referred to as G5* and G2* respectively. The predicted structure of the putative GTPase based on consensus motifs from the Ylqf/YawG family show the six-stranded β-sheet of the G-domain wherein the conserved sequence elements, G5*-G4-G1-G3 stack almost perfectly over each other (FIG. 1C). Interestingly, except for a bacterial ancestor of this group of GTPases, YjeQ (Daigle et al., 2002) none of the eukaryotic members shown in FIGS. 1A and B have proven GTPase activity.

### Example 3

### Grn1 is Required for Optimal Growth of S. pombe and Localizes to the Nucleolus

In order to study the function of this putative GTPase, the ORF SPBC26H8.08c was deleted and replaced with the Ura4 gene using a previously described PCR-based deletion strategy (Bahler et al., 1998; Chen et al., 2004). Dissection of tetrads from at least 24 independent asci yielded two of the four spores from each tetrad that grew extremely slowly on YES medium at 32° (FIG. 2A) when compared with the wild type strain and similarly at 24° or 37° C. (results not shown). These slow-growing colonies were confirmed as having the SPBC26H8.08c deletion. In contrast, genes encoding similar putative GTPases from yeast, Nug1 (YER006W) and Nug2lNog2 (YNR053C) (Bassler et al., 2001; Saveanu et al., 2001) are essential for viability.

To investigate the subcellular localization of the Grn1p, its ORF Grn1 was cloned as a C-terminal fusion to the green fluorescent protein (GFP) downstream from an inducible promoter, nmt1 and transformed into the null mutant. FIG. 2B shows that the growth phenotype of the null mutant was rescued. To establish that Grn1p localizes to the nucleolus, we used as a nucleolar reference marker, Fibrillarin/Nop1p (Aris and Blobel, 1988; Henriquez et al., 1990). Monoclonal antibody staining detected Fibrillarin/Nop1p in a discrete region (selectively excluding the DAPI-stained area) of the nucleus (FIG. 2C). Grn1p:GFP, localizes primarily to the nucleolus in S. pombe as seen by its co-localization with Fibrillarin/Nop1p (FIG. 2C).

Since the episomally expressed gene is able to complement the growth defect in a null mutant, we confirm the involvement of this nucleolar protein in growth. In addition to Grn1, S. pombe has at least three other ORFs predicted to generate putative nuclear/nucleolar GTPases with an HSR1_MMR1-type domain (FIG. 1B). We conclude the function of the protein encoded by Grn1 does not completely overlap with the other three putative GTPases.

Although our genetic data implies that the gene is not essential for viability, we did however observe that 20-40% of cells exhibited morphogenic aberrations represented by irregular, uneven or over-deposition of septum material as well as septation and cell separation defects often resulting in pseudo-filamentous or multi-septated cells (FIG. 2D), suggesting a failure of cytokinesis in those cells.

### Example 4

### Pre-rRNA Processing is Impaired in Grn1-Depleted Cells

The yeast rDNA unit is made up of the 35S pre-rRNA operon and two non-transcribed spacers interrupted by the 5S rRNA gene. The 35S pre-rRNA operon, flanked on either end by externally transcribed spacers 5′-ETS and 3′-ETS, eventually gives rise to the mature 18S, 5.8S and 25S rRNA species (Venema and Tollervey, 1999).

The S. pombe rRNA processing pathway (FIG. 3B) is similar to that of S. cerevisiae and several other eukaryotes although it may depart from the same in specific processing steps (Good et al., 1997). Since the S. cerevisiae GTPases Nug1p and Nug2p were linked closely with pre-rRNA processing (Bassler et al., 2001; Saveanu et al., 2001), we asked if the grn1Δ mutant was defective in the processing of 35S pre-rRNA precursor to mature rRNA species by performing a northern-blot analysis. Probes corresponding to the 5′ ETS, 5.8S, ITS1 and ITS2 regions are indicate in FIG. 3B.

Our 5′ETS probe beginning at −900 bp upstream of the 5′ end of 18SrRNA covers all the putative 5′ETS processing sites and would thus identify all intermediates from 35S to 32S pre-rRNA (it will not detect 32S) species. The ITS1 probe (D→A2) identifies 35S, 32S and 20S whereas ITS1 (A3→B1) identifies 35S, 32S, 27S′. The ITS2 (E→C1) probe detects 35S, 32S, 27S′, 27S and 7S species. 27S′, 27S are indistinguishable (Good et al., 1997).

To detect 5.8S mature rRNA, the probe was based on a sequence within the 5.8S operon. The results depicted in FIG. 3B, show the accumulation of the 35S pre-rRNA species in the null mutant when compared to the wild type with a concomitant decrease in the 25S, 18S and 5.8S mature rRNA species. Under wild type conditions it may not be possible to see the 35S pre-rRNA species since it is processed very rapidly (Good et al., 1997; Venema and Tollervey, 1999).

The observations that mature rRNA species in the null mutant are significantly lower than that of the wild type, coupled with the increase in levels of the 35S pre-rRNA species is suggestive of a significant inhibition or slowing down of the early pre-rRNA processing steps. Accumulation of 35S pre-rRNA was observed for ts mutants of nug1 and nug2 (Bassler et al., 2001) and like those investigators, we cannot rule out the possibility that the early processing phenotype we observe may be a consequence of the reduced growth rate rather than a direct effect on rRNA processing. However, in another study (also in S. cerevisiae) depletion of the ribosomal protein L25 (Rpl25) led to a severe reduction in the levels of the large sub-unit rRNAs (25S, 18S and 5.8S) with a concomitant accumulation of the 35S-pre-rRNA (van Beekvelt et al., 2001).

Interestingly, the same study also established a similar phenotype if binding of Rpl25 to the pre-ribosome was abolished suggesting that the assembly of Rpl25 with the 60S pre-ribosome is required for rRNA processing Rpl25 is incorporated into nascent pre-60S ribosomes and is known to bind both, 35S pre-rRNA as well as the 25S rRNA in yeast (el-Baradi et al., 1987; van Beekvelt et al., 2001; van Beekvelt et al., 2000). Furthermore, nuclear or nucleolar retention of Rpl25 has been observed for mutants defective in 60S ribosome biogenesis and/or nucleocytoplasmic transport (Bassler et al., 2001; Saveanu et al., 2001; Strasser and Hurt, 1999; Tschochner and Hurt, 2003).

### Example 5

### Grn1p is Required for Nuclear Export of the Putative Ribosomal Protein Rpl25a

RNA export from the nucleus is linked to its proper processing and packaging into ribonucleoprotein complexes within the nucleus (Strasser and Hurt, 1999; Tschochner and Hurt, 2003). The use of functional GFP-tagged ribosomal protein reporters has greatly facilitated the elucidation of the large-subunit (Rpl25:GFP, Rpl11:GFP) (Gadal et al., 2001; Hurt et al., 1999; Stage-Zimmermann et al., 2000) and small-subunit (Rps2:GFP) (Grandi et al., 2002; Milkereit et al., 2003) ribosome assembly and nucleolar/nuclear export pathway.

L25 (Rpl25 in yeast and L23 in plant/mammal/human) is perhaps the most extensively studied and highly conserved eukaryotic ribosomal protein (r-protein) (FIG. 9). Rpl25 may be among the first proteins to assemble into the pre-ribosome binding to either the 35S pre-rRNA and/or 26S-rRNA and is essential for the production of mature 25S rRNA species in yeast (el-Baradi et al., 1987; van Beekvelt et al., 2001; van Beekvelt et al., 2000). We therefore used an in vivo assay exploiting the green fluorescent protein (GFP)-tagged version of the S. pombe putative ribosomal protein RpL25a encoded by the ORF SPBC106.18 that is homologous to ORF YOL127Wof S. cerevisiae encoding Rpl25 (FIG. 9).

The S. cerevisiae RpL25:GFP, binds to pre-rRNA, assembles with 60S ribosomal subunits after its import into the nucleolus and is subsequently exported into the cytoplasm, thus allowing for monitoring of the localization of pre-60S and 60S particles by fluorescence microscopy (Hurt et al., 1999). Since nuclear retention of 60S subunit/Rpl25-GFP was reported for mutants of nug1 and nug2 (Bassler et al., 2001; Saveanu et al., 2001) and it is predicted that such a phenotype may stem from an inability to process rRNA properly resulting in ribosome maturation defects (Tschochner and Hurt, 2003), we cloned the S. pombe putative Rpl25a into a vector and expressed nmt1:Rpl25a:GFP in wild type (Grn1) and null mutant (Δgrn1) strains.

When induced (nmt1 ON), Rpl25a:GFP was detected primarily at the nuclear pore complexes (NPC staining) of the wild type (FIG. 4). However, in stark contrast, the null mutant consistently revealed a nuclear accumulation of RpL25a:GFP with a higher proportion within the nucleolus (FIG. 4, panels showing enlarged nucleus). The combination of our results regarding the impaired nuclear export of Rpl25a:GFP in the grn1Δ mutant coupled with its inability to efficiently process the 35S pre-rRNA transcript is suggestive of a two-fold role. One, in early 5′-end pre-rRNA processing step(s) and two, in the assembly and export of Rpl25a/pre-ribosomal complexes from the nucleus.

### Example 6

### The Canonical G Domain and a Putative RNA-Binding Domain (RG) are Required for Grn1p Function

To dissect the molecular mechanism underlying Grn1p function and to assess the functional significance of the signature GTP-binding motifs and the RG domain, we tested the ability of Grn1p with deletions of those motifs/domains to complement the growth defect of the null mutant.

Constructs were engineered so that C-terminal GFP fusion proteins were generated with deletions of the putative coiled-coil domain ΔCC (AA70-90), ΔG5* (M164-M175), ΔG4 (M195-AA208), ΔG1 (AA276-M283), ΔG3 (AA326-AA329) and the RNA binding domain ARG, (AA405415) (FIG. 5A).

The grn1Δ strain was transformed with plasmids bearing the above constructs (Table 2).

An nmt1 promoter drove transcription of wild type and mutant versions of Grn1 in the absence of thiamine as described below. Independent transformants were struck for single colonies onto selective growth medium allowing for induction of gene expression. FIG. 5A depicts growth of the various mutants in comparison to the wild type and null mutant. The WT and the ACC mutant fully complement the null mutant. However, the ΔG5, ΔG4, ΔG1, ΔG3 and ARG mutants were unable to rescue the null growth defect indicating that those domains or motifs were required for its function.

We noted that even in the presence of thiamine (nmt1 turned off), WT and ΔCC grew very well. The nmt1 promoter is known to be ‘leaky’ and as a result, even a low expression is sufficient to rescue growth. Expression of WT and mutant GFP-tagged proteins was verified by western analysis using anti-GFP (FIG. 5B) after which the membrane was stripped and re-probed with actin antibody to visualize actin levels. FIG. 5B shows that levels of ΔG5, ΔG4, ΔG1, ΔG3 and ΔRG deletion proteins are extremely low when compared with the WT or ΔCC levels of expression indicating the proteins may be unstable or unable to fold.

Clearly, deletion of any one of the ΔG- or -ΔRG motifs results in a null phenotype and that the G5, G4, G1, G3 and RG motifs are equally critical for Grn1p function. The quantity and size of each mutant protein was also tested by in vitro transcription-translation from a rabbit reticulocyte system. FIG. 5C shows an equivalent amount of each protein was realized. In each case, doublet bands were evident (including wild type) which were not present in the null mutant. Similar doublets were also observed in the westerns in FIG. 5B which was unrelated to the mutations introduced into the protein.

### Example 7

### Mutations in the Functional Domains of Grn1p Alter its Localization within the Nucleus

In FIG. 2C, we established that full-length Grn1p:GFP localized to the nucleolus. We wanted to know whether Grn1p function was related to its nucleolar localization and if the ΔAG5, ΔG4, ΔG1, ΔG3 and ARG deletions in fact, were mislocalized, thereby unable to rescue the null phenotype. GFP localization data is depicted in FIG. 6A. The ΔCC mutant localized to the nucleolus like the wild type pictured in FIG. 2C whereas ΔG1, ΔG3, ΔG4, ΔG5 and ARG were all excluded from the nucleolus. Each exhibited a clear aggregation of GFP signal at what appears to be a well-defined border between the nuclear and nucleolar region (FIG. 6A-B). In contrast, NS mutants lacking the same motifs exhibited irregular aggregates (Tsai and McKay, 2002; Tsai and McKay, 2005). Similar to NS, we did however find a distortion of nucleolar/nuclear morphology with the ΔG-motifs and ARG mutants as compared to the WT (FIG. 2C), or ΔCC (FIG. 6A). Thus, as for NS, mutations in Grn1p that affect its normal localization also disrupt nucleolar stability and integrity.

As depicted in the cartoon (FIG. 6C), mammalian nucleoli contain fibrillar centers (FC) known to house rDNA genes, surrounded by a layer called the dense fibrillar component (DFC) in which the maturation of pre-rRNA transcripts is said to take place which is in-turn, surrounded by a granular component (GC) wherein the assembly of pre-ribosomes takes place (Carmo-Fonseca et al., 2000).

Similar morphological nucleolar subcompartments are found in S. pombe (Leger-Silvestre et al., 1997) and S. cerevisiae (Trumtel et al., 2000). Though presence of FC and DFC in yeast is currently a debated issue, the existence of a granular zone is accepted as comprising pre-ribosomes/ribosomes (Thiry and Lafontaine, 2005).

Thus, accumulation of GFP signal at the GC region and compared with nucleolar accumulation in a ΔCC mutant as shown in FIG. 6B (here, ΔG5 is used as a representative since all of them have a similar phenotype). suggests that the absence of G- or RG motifs may block release from the pre-ribosome assembly thereby restricting cycling of the GTPase in and out of the nucleolar compartment or, that movement of preribosomes from the nucleolus-nucleus interface to the NPC is compromised.

Since wild type and the ACC mutant proteins are nucleolar and the strains do not have a growth defect we must conclude from FIGS. 4 and 5 that failure of Grn1p to localize or relocate to the nucleolus is responsible for the null phenotype and that nucleolar localization was indeed essential for Grn1p function. Yet, studies with NS suggest that there exists a dynamic partitioning of the protein between the nucleolus and nucleoplasm (Tsai and McKay, 2002; Tsai and McKay, 2005) possibly, the driving force behind signal-mediated activities within nuclear subcompartments (Misteli, 2005).

It would be difficult to reconcile our rRNA processing and Rpl25 data with the concept of ‘nucleolar retention rather than release’, as being the cornerstone of Grn1p activity as has been suggested for NS (Misteli, 2005) since it would be necessary for the putative GTPase to be physically present within the nucleolus to execute such functions.

### Example 8

### A Human Homolog FLJ10613 (GNL3L) Encoding a Hypothetical Protein Rescues the grn1Δ Growth Defect in S. pombe

As mentioned earlier, BLASTp and CD-search searches of available databases identified several HSR1_MMR1 GTP-binding proteins similar to Grn1p (FIG. 1A-C). However, our attention was drawn to the association of some of these human proteins with cancer. Ngp1 (GNL2) was identified as a nucleolar breast tumor-associated autoantigen (Racevskis et al., 1996). NS, a nucleolar GTPase controlling stems cell proliferation was found in several cancer cell lines (Liu et al., 2004; Sijin et al., 2004; Tsai and McKay, 2002; Tsai and McKay, 2005).

However, GNL3L (also referred to as FLJ10613) is essentially an uncharacterized and hypothetical protein predicted to be a GTPase (Ota et al., 2004). Interestingly enough, of the 700-or-so human nucleolar proteins in the nucleolar protein database (http://lamondlab.com/nopdb/) only the above three, Ngp1 (GNL2), GNL3L, NS and a fourth, GNL-1 possess the G5* motif-[DARDP] (FIG. 1B). According to AceView (http://www.ncbi.nlm.nih.gov/IEB/Research/Acembly), these genes are expressed at very high (4.1 times; Ngp1), high (3.2 times; GNL3L), or well expressed (0.8 times; NS) the average gene (GNL-1 expression is moderate to low).

We noted that although all these proteins were expressed as well in fission yeast, NGP-1, NS or the yeast ScNug1 were unable to complement SpGrn1 activity when induced. Surprisingly however, we observed that NGP-1 was able to fully complement SpGrn1 when induced very weakly (nmt1 OFF, FIG. 7A) implying that nmt1-dependent overexpression of NGP-1 was toxic to cell growth.

GNL3L maps on chromosome X at Xp11.22. Two major nucleolar proteomic analyses (Andersen et al., 2002; Scherl et al., 2002) failed to identify GNL3L. In the Nucleolar Proteome Database GNL3L appears only as a hypothetical component of the nucleolus based on SILAC analysis (Stable isotope Labeling by Amino acids in Cell culture) (http://lamondlab.com/nopdb/).

To determine whether GNL3L could complement the growth defect in the S. pombe grn1Δ mutant, we cloned its ORF from a HeLa cell cDNA library into an inducible (nmt1) yeast expression vector and assayed growth of the yeast transformants (FIG. 7A). GNL3L complemented the grn1Δ deletion in a manner similar to that of Grn1.

Similarly, the null growth phenotype is rescued when the GNL3L is expressed from the endogenous Grn1 promoter. Here, the Grn1 portion of the integrated genomic copy of Grn1:FLAG was replaced with the GNL3L-ORF so the latter would be transcribed from the S. pombe native promoter. Thus, GNL3L:FLAG (YNB858) and Grn1:FLAG (YNB859) are isogenic.

FIG. 7B shows that though GNL3L:FLAG exhibited a longer lag, its growth rate became almost equal to that of Grn1. Thus, under identical endogenous promoter activities, GNL3L expression complements the Grn1 deletion.

In order to test whether GNL3L was also a nucleolar protein and that fission yeast Grn1p:GFP would similarly be targeted to the nucleolus in human cells, Cos-7 cells were tranfected with GNL3L:GFP or Grn1p:GFP. The human nucleolar protein Nucleolin was used as a marker for the nucleolus. FIG. 7C shows that both the GTPases are targeted to the mammalian nucleolus. Our genetic complementation thus identifies GNL3L as a homolog of Grn1p. FIG. 7D show the localization of GNL3L:FLAG in S. pombe. Comparing the localization of fibrillarin and GNL3L (FIG. 7D, see arrow), we noted the latter was not concentrated in the nucleolus to the same degree as Grn1:GFP (compared with FIG. 2C) suggesting that GNL3L and Grn1p may differ in their ability to either be targeted to, or retained by, the nucleolus.

### Example 9

### GNL3L Functionally Complements Grn1p

Since GNL3L complemented the Grn1Δ growth defect and the GFP-tagged protein localized to the nucleolus, we wanted to know if its expression in fission yeast would (a) rescue the rRNA processing defect and prevent the accumulation of the 35S pre-rRNA and (b) rescue the Rpl25:GFP nuclear export defect.

Strains expressing GNL3I:FLAG or Grn1:FLAG from the endogenous Grn1 promoter and the null mutant were investigated for rRNA processing using 5′ETS, 5.8S, ITS1 and ITS2 probes. FIG. 3 shows that in the GNL3L:FLAG strain there is a marked reduction in accumulation of 35S pre-rRNA accompanied by a significant increase in the amounts of 18S and 25S mature rRNA species when compared with the null mutant. ITS1 and ITS2 probing confirmed the reduction in accumulation of 35S pre-rRNA when GNL3L was expressed. Thus, expression of GNL3L rescues the 5′-pre-rRNA processing defect in the null mutant although it was not equivalent to the wild type.

Since nuclear export of Rpl25a:GFP was blocked in the null mutant (FIG. 4) we asked whether GNL3L could rescue that ribosome export defect. As shown in FIG. 4, Rpl25a:GFP accumulated within the nuclei (nucleoli) of the grn1 null but is exported to the nuclear periphery and cytoplasm with equal efficiency in both GNL3L and Grn1p strains. Thus, GNL3L functionally complements Grn1p although it did not completely rescue the RNA processing defect. Given it restored Rpl25a export and growth close to wild type levels, one possibility is that the primary defect in the Grn1p null mutant is a reduced efficiency in 60S/Rpl25a export that results in uncoupling ribosomal subunit export from upstream rRNA processing events.

Expression of Grn1p completely restores the connectivity between the two processes whereas GNL3L only partially does so. A contributing factor for the partial rescue could be the altered nuclear localization in fission yeast that we noted for GNL3L. Although the G-domain regions of Grn1p, GNL3L and NS are very similar, they differ moderately at the N-terminal and quite significantly at the C-terminal end (FIG. 10). It is known that the nucleolar localization and nuclear shuttling of NS is dependent on its N-terminal basic domain and regulation of the latter by its G1/GTP-binding state (Tsai and McKay, 2002; Tsai and McKay, 2005).

Preliminary analysis on the N-terminus of Grn1p identified a putative nuclear/nucleolar sequence similar but not identical to either GNL3L or NS, which when deleted failed to concentrate Grn1p:GFP in the nucleolus (FIG. 9). Quite surprisingly, these mutants did not exhibit any growth defect compared with either the null mutant or G-motif/RG mutants tested. Absence of nucleolar sequestration may thus allow these cells to ‘override’ the wild type requirement for this particular pathway.

Conversely, as envisaged for NS by Tsai and McKay (Tsai and McKay, 2005), and Misteli (Misteli, 2005), the function(s) of Grn1p are realized in shuttling between the nucleolus/nucleoplasm interface (GC) and the nucleoplasm. Thus, in the absence of any one of the G-motifs or RG, Grn1 activity was impeded at the GC as shown in FIG. 6B whereas, deletion of the putative targeting sequence allowed some of the protein to be retained in the nucleoplasm where it could continue to function.

### Example 10

### GNL3L is Required for Proliferation of Mammalian Cells

Our results imply an important and unique role for the putative GTPase Grn1p in fission yeast since it is required for wild type growth despite the presence of three other HSR1_MMR1 putative nucleolar GTPases (see FIG. 1B).

In human cells, depletion or overexpression of NS causes a reduction in cell proliferation in CNS stem cells and transformed cells (Tsai and McKay, 2002). In another study, HeLa cells wherein NS expression was knocked down with small inhibitory RNA (siRNA), could not complete DNA synthesis to pass through S phase resulted in an increase of cells in the G0/G1 phase (Sijin et al., 2004). We also investigated if decreasing expression of GNL3L would affecting proliferation in HeLa cells.

HeLa cells were tranfected with GNL3L-siRNA, a scrambled version of the GNL3L-siRNA, Luciferase-specific siRNA and an empty vector, pcDNA3. Cultures transfected with GNL3L-siRNA showed consistently a 30-40% decrease in number of cells when compared with Luciferase siRNA or GNL3L non-specific (scrambled sequence) siRNA-tranfected cells used as negative controls (FIG. 8A). RT-PCR analysis using primers specific for a 600 bp GNL3L-specific product or 460 bp actin-specific product confirmed a reduction in GNL3L RNA in cultures treated with GNL3L-specific siRNA when compared with cultures transfected with control siRNA (FIG. 8B). Similar levels of a ‘house-keeping’ gene, actin-specific PCR product from all above siRNA treatments ensured there was no bias for either RNA or the RT-PCR reaction.

We thus demonstrate that the specific knockdown of GNL3L expression is consistent with a decrease in HeLa cell proliferation and is also consistent with the growth function of its homolog Grn1p in S. pombe.

We examined three GTPases, NGP-1, NS and GNL3L, for their ability to complement Grn1. Despite the fact that all three were expressed in S. pombe, only GNL3L rescued the grn1 mutant. The S. cerevisiae Nug1 did so only very weakly. NGP-1 complemented Grn1 at low levels of induction (nmt1 OFF) but inhibited growth at high levels of expression (nmt1 ON). Our work clearly demonstrates that NS does not complement Grn1.

The examples of the present invention demonstrate that the putative nucleolar GTPase, Grn1p or its human homolog, GNL3L is required for normal growth despite the presence of multiple HSR1_MMR1-type GTP-binding nucleolar GTPases underscoring their unique or specific importance. Should it be that differences in these GTPase activities are related to sub-nucleolar/nuclear compartmentalization, their locales must then define specific metabolic areas (or functions) within the nucleolus. For example, high-resolution electron spectroscopic imaging studies recently revealed that NS localizes to the GC regions of the nucleolus having little or no rRNA thus leading to the prediction that NS may not be associated with ribosome biogenesis/rRNA processing (Politz et al., 2005).

In the present invention, we showed that G-motif and the RG mutants of Grn1p assemble at the nucleolus/nuclear boundary, clearly a well-defined region, emphasizing the presence of a definitive infrastructure rather than the lack of one as previously believed (see Carmo-Fonseca et al., 2000; Thiry and Lafontaine, 2005). Thus, nucleolar GTPases help to shed light on key sites of non-ribosomal or ribosomal activity in the nucleolus and their respective roles in growth. By using methods well known to the person skilled in the art, this knowledge can be readily applied as methods to control proliferation of cells, particular cancerous cells.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

### REFERENCES

Altschul, S. F., Madden, T. L., Schaffer, A. A., Zhang, J., Zhang, Z., Miller, W. and Lipman, D. J. (1997) Gapped BLAST and PSI-BLAST: a new generation of protein database search programs. Nucleic Acids Res, 25, 3389-3402.
Andersen, J. S., Lyon, C. E., Fox, A. H., Leung, A. K., Lam, Y. W., Steen, H., Mann, M. and Lamond, A. I. (2002) Directed proteomic analysis of the human nucleolus. Curr Biol, 12, 1-11.
Aris, J. P. and Blobel, G. (1988) Identification and characterization of a yeast nucleolar protein that is similar to a rat liver nucleolar protein. J Cell Biol, 107, 17-31.
Bahler, J., Wu, J. Q., Longtine, M. S., Shah, N. G., McKenzie, A., 3rd, Steever, A. B., Wach, A., Philippsen, P. and Pringle, J. R. (1998) Heterologous modules for efficient and versatile PCR-based gene targeting in Schizosaccharomyces pombe. Yeast, 14, 943-951.
Balasundaram, D., Benedik, M. J., Morphew, M., Dang, V. D. and Levin, H. L. (1999) Nup124p is a nuclear pore factor of Schizosaccharomyces pombe that is important for nuclear import and activity of retrotransposon Tf1. Mol Cell Biol, 19, 5768-5784.
Bassler, J., Grandi, P., Gadal, O., Lessmann, T., Petfalski, E., Tollervey, D., Lechner, J. and Hurt, E. (2001) Identification of a 60S preribosomal particle that is closely linked to nuclear export. Mol Cell, 8, 517-529.
Bateman, A., Coin, L., Durbin, R., Finn, R. D., Hollich, V., Griffiths-Jones, S., Khanna, A., Marshall, M., Moxon, S., Sonnhammer, E. L., Studholme, D. J., Yeats, C. and Eddy, S. R. (2004) The Pfam protein families database. Nucleic Acids Res, 32, D138-141.
Bernardi, R. and Pandolfi, P. P. (2003) The nucleolus: at the stem of immortality. Nat Med, 9, 24-25.
Carmo-Fonseca, M., Mendes-Soares, L. and Campos, I. (2000) To be or not to be in the nucleolus. Nat Cell Biol, 2, E107-112.
Chen, X. Q., Du, X., Liu, J., Balasubramanian, M. K. and Balasundaram, D. (2004) Identification of genes encoding putative nucleoporins and transport factors in the fission yeast Schizosaccharomyces pombe: a deletion analysis. Yeast, 21, 495-509.
Daigle, D. M., Rossi, L., Berghuis, A. M., Aravind, L., Koonin, E. V. and Brown, E. D. (2002) YjeQ, an essential, conserved, uncharacterized protein from Escherichia coli, is an unusual GTPase with circularly permuted G-motifs and marked burst kinetics. Biochemistry, 41, 11109-11117.
el-Baradi, T. T., de Regt, V. C., Planta, R. J., Nierhaus, K. H. and Raue, H. A. (1987) Interaction of ribosomal proteins L25 from yeast and EL23 from E. coli with yeast 26S and mouse 28S rRNA. Biochimie, 69, 939-948.
Fatica, A. and Tollervey, D. (2002) Making ribosomes. Curr Opin Cell Biol, 14, 313-318.
Gadal, O., Strauss, D., Kessl, J., Trumpower, B., Tollervey, D. and Hurt, E. (2001) Nuclear export of 60s ribosomal subunits depends on Xpo1p and requires a nuclear export sequence-containing factor, Nmd3p, that associates with the large subunit protein Rpl10p. Mol Cell Biol, 21, 3405-3415.
Good, L., Intine, R. V. and Nazar, R. N. (1997) The ribosomal-RNA-processing pathway in Schizosaccharomyces pombe. Eur J Biochem, 247, 314-321.
Grandi, P., Rybin, V., Bassler, J., Peffalski, E., Strauss, D., Marzioch, M., Schafer, T., Kuster, B., Tschochner, H., Tollervey, D., Gavin, A. C. and Hurt, E. (2002) 90S pre-ribosomes include the 35S pre-rRNA, the U3 snoRNP, and 40S subunit processing factors but predominantly lack 60S synthesis factors. Mol Cell, 10, 105-115.
Henriquez, R., Blobel, G. and Aris, J. P. (1990) Isolation and sequencing of NOP1. A yeast gene encoding a nucleolar protein homologous to a human autoimmune antigen. J Biol Chem, 265, 2209-2215.
Hermeking, H., Lengauer, C., Polyak, K., He, T. C., Zhang, L., Thiagalingam, S., Kinzler, K. W. and Vogelstein, B. (1997) 14-3-3 sigma is a p53-regulated inhibitor of G2/M progression. Mol Cell, 1, 3-11.
Hurt, E., Hannus, S., Schmelzl, B., Lau, D., Tollervey, D. and Simos, G. (1999) A novel in vivo assay reveals inhibition of ribosomal nuclear export in ran-cycle and nucleoporin mutants. J Cell Biol, 144, 389-401.
Lee, M. and Nurse, P. (1988) Cell cycle control genes in fission yeast and mammalian cells. Trends Genet, 4, 287-290.
Lee, M. G. and Nurse, P. (1987) Complementation used to clone a human homologue of the fission yeast cell cycle control gene cdc2. Nature, 327, 31-35.
Leger-Silvestre, I., Noaillac-Depeyre, J., Faubladier, M. and Gas, N. (1997) Structural and functional analysis of the nucleolus of the fission yeast Schizosaccharomyces pombe. Eur J Cell Biol, 72, 13-23.
Leipe, D. D., Wolf, Y. I., Koonin, E. V. and Aravind, L. (2002) Classification and evolution of P-loop GTPases and related ATPases. J Mol Biol, 317, 41-72.
Liu, S. J., Cai, Z. W., Liu, Y. J., Dong, M. Y., Sun, L. Q., Hu, G. F., Wei, Y. Y. and Lao, W. D. (2004) Role of nucleostemin in growth regulation of gastric cancer, liver cancer and other malignancies. World J Gastroenterol, 10, 1246-1249.
Marchler-Bauer, A., Anderson, J. B., Cherukuri, P. F., DeWeese-Scott, C., Geer, L. Y., Gwadz, M., He, S., Hurwitz, D. I., Jackson, J. D., Ke, Z., Lanczycki, C. J., Liebert, C. A., Liu, C., Lu, F., Marchler, G. H., Mullokandov, M., Shoemaker, B. A., Simonyan, V., Song, J. S., Thiessen, P. A., Yamashita, R. A., Yin, J. J., Zhang, D. and Bryant, S. H. (2005) CDD: a Conserved Domain Database for protein classification. Nucleic Acids Res, 33, D192-196.
Milkereit, P., Gadal, O., Podtelejnikov, A., Trumtel, S., Gas, N., Peffalski, E., Tollervey, D., Mann, M., Hurt, E. and Tschochner, H. (2001) Maturation and intranuclear transport of pre-ribosomes requires Noc proteins. Cell, 105, 499-509.
Milkereit, P., Strauss, D., Bassler, J., Gadal, O., Kuhn, H., Schutz, S., Gas, N., Lechner, J., Hurt, E. and Tschochner, H. (2003) A Noc complex specifically involved in the formation and nuclear export of ribosomal 40 S subunits. J Biol Chem, 278, 4072-4081.
Misteli, T. (2005) Going in GTP cycles in the nucleolus. J Cell Biol, 168, 177-178.
Moreno, S., Klar, A. and Nurse, P. (1991) Molecular genetic analysis of fission yeast Schizosaccharomyces pombe. Methods Enzymol, 194, 795-823.
Nissan, T. A., Bassler, J., Peffalski, E., Tollervey, D. and Hurt, E. (2002) 60S pre-ribosome formation viewed from assembly in the nucleolus until export to the cytoplasm. Embo J, 21, 5539-5547.
Normile, D. (2002) Cell proliferation. Common control for cancer, stem cells. Science, 298, 1869.
Nurse, P. (1997) The Josef Steiner Lecture: CDKs and cell-cycle control in fission yeast: relevance to other eukaryotes and cancer. Int J Cancer, 71, 707-708.
Ota, T., et al., (2004) Complete sequencing and characterization of 21,243 full-length human cDNAs. Nat Genet, 36, 40-45.
Politz, J. C., Polena, I., Trask, I., Bazett-Jones, D. P. and Pederson, T. (2005) A nonribosomal landscape in the nucleolus revealed by the stem cell protein nucleostemin. Mol Biol Cell, 16, 3401-3410.
Racevskis, J., Dill, A., Stockert, R. and Fineberg, S. A. (1996) Cloning of a novel nucleolar guanosine 5′-triphosphate binding protein autoantigen from a breast tumor. Cell Growth Differ, 7, 271-280.
Rustici, G., Mata, J., Kivinen, K., Lio, P., Penkett, C. J., Burns, G., Hayles, J., Brazma, A., Nurse, P. and Bahler, J. (2004) Periodic gene expression program of the fission yeast cell cycle. Nat Genet, 36, 809-817.
Saveanu, C., Bienvenu, D., Namane, A., Gleizes, P. E., Gas, N., Jacquier, A. and Fromont-Racine, M. (2001) Nog2p, a putative GTPase associated with pre-60S subunits and required for late 60S maturation steps. Embo J, 20, 6475-6484.
Scherl, A., Coute, Y., Deon, C., Calle, A., Kindbeiter, K., Sanchez, J. C., Greco, A., Hochstrasser, D. and Diaz, J. J. (2002) Functional proteomic analysis of human nucleolus. Mol Biol Cell, 13, 4100-4109.
Sheldrick, K. S. and Carr, A. M. (1993) Feedback controls and G2 checkpoints: fission yeast as a model system. Bioessays, 15, 775-782.
Sijin, L., Ziwei, C., Yajun, L., Meiyu, D., Hongwei, Z., Guofa, H., Siguo, L., Hong, G., Zhihong, Z., Xiaolei, L., Yingyun, W., Yan, X. and Weide, L. (2004) The effect of knocking-down nucleostemin gene expression on the in vitro proliferation and in vivo tumorigenesis of HeLa cells. J Exp Clin Cancer Res, 23, 529-538.
Stage-Zimmermann, T., Schmidt, U. and Silver, P. A. (2000) Factors affecting nuclear export of the 60S ribosomal subunit in vivo. Mol Biol Cell, 11, 3777-3789.
Strasser, K. and Hurt, E. (1999) Nuclear RNA export in yeast. FEBS Lett, 452, 77-81.
Takai, Y., Sasaki, T. and Matozaki, T. (2001) Small GTP-binding proteins. Physiol Rev, 81, 153-208.
Tatusov, R. L., Galperin, M. Y., Natale, D. A. and Koonin, E. V. (2000) The COG database: a tool for genome-scale analysis of protein functions and evolution. Nucleic Acids Res, 28, 33-36.
Thiry, M. and Lafontaine, D. L. (2005) Birth of a nucleolus: the evolution of nucleolar compartments. Trends Cell Biol, 15, 194-199.
Trumtel, S., Leger-Silvestre, I., Gleizes, P. E., Teulieres, F. and Gas, N. (2000) Assembly and functional organization of the nucleolus: ultrastructural analysis of Saccharomyces cerevisiae mutants. Mol Biol Cell, 11, 2175-2189.
Tsai, R. Y. and McKay, R. D. (2002) A nucleolar mechanism controlling cell proliferation in stem cells and cancer cells. Genes Dev, 16, 2991-3003.
Tsai, R. Y. and McKay, R. D. (2005) A multistep, GTP-driven mechanism controlling the dynamic cycling of nucleostemin. J Cell Biol, 168, 179-184.
Tschochner, H. and Hurt, E. (2003) Pre-ribosomes on the road from the nucleolus to the cytoplasm. Trends Cell Biol, 13, 255-263.
van Beekvelt, C. A., de Graaff-Vincent, M., Faber, A. W., van't Riet, J., Venema, J. and Raue, H. A. (2001) All three functional domains of the large ribosomal subunit protein L25 are required for both early and late pre-rRNA processing steps in Saccharomyces cerevisiae. Nucleic Acids Res, 29, 5001-5008.
van Beekvelt, C. A., Kooi, E. A., de Graaff-Vincent, M., Riet, J., Venema, J. and Raue, H. A. (2000) Domain III of Saccharomyces cerevisiae 25 S ribosomal RNA: its role in binding of ribosomal protein L25 and 60 S subunit formation. J Mol Biol, 296, 7-17.
Varadarajan, P., Mahalingam, S., Liu, P., Ng, S. B., Gandotra, S., Dorairajoo, D. S. and Balasundaram, D. (2005) The Functionally Conserved Nucleoporins Nup124p from Fission Yeast and the Human Nupl53 Mediate Nuclear Import and Activity of the Tf1 Retrotransposon and HIV-1 Vpr. Mol Biol Cell, 16, 1823-1838.
Venema, J. and Tollervey, D. (1999) Ribosome synthesis in Saccharomyces cerevisiae. Annu Rev Genet, 33, 261-311.
Vernet, C., Ribouchon, M. T., Chimini, G. and Pontarotti, P. (1994) Structure and evolution of a member of a new subfamily of GTP-binding proteins mapping to the human MHC class I region. Mamm Genome, 5, 100-105.

1. An isolated polypeptide comprising an amino acid sequence at least 85% homologous to SEQ ID NO: 2 or a conservative variant thereof, wherein the polypeptide regulates proliferation of a cell.

2. An isolated polynucleotide, wherein the polynucleotide encodes the polypeptide of claim 1.

3. The isolated polynucleotide according to claim 2, wherein the polynucleotide comprises the nucleotide sequence of SEQ ID NO: 1.

4. An isolated a polypeptide comprising an amino acid sequence at least 85% homologous to SEQ ID NO: 4 or a conservative variant thereof, wherein the polypeptide regulates proliferation of a cell.

5. An isolated polynucleotide, wherein the polynucleotide encodes the polypeptide of claim 4.

6. The isolated polynucleotide according to claim 5, wherein the polynucleotide comprises the nucleotide sequence of SEQ ID NO: 3.

7. The isolated polynucleotide of claim 2, wherein the polynucleotide is comprised in a vector.

8. The isolated polynucleotide according to claim 2, wherein the polynucleotide is transfected into an isolated host cell.

9. The isolated host cell of claim 8, wherein the host cell is selected from the group consisting of an eukaryotic cell and a prokaryotic cell.

10. A method for inhibiting proliferation of a cell, comprising altering the level of a polypeptide comprising an amino acid sequence at least 85% homologous to SEQ ID NO: 2 in the cell, thereby inhibiting proliferation of the cell.

11. The method according to claim 10, wherein the polypeptide comprises the amino acid sequence of SEQ ID NO: 2.

12. The method according to claim 10, wherein altering the level of the polypeptide comprises decreasing the level of the polypeptide.

13. The method of claim 10, wherein altering the level of the polypeptide comprises decreasing transcription of a nucleic acid sequence encoding the polypeptide.

14. The method according to claim 12, wherein altering the level of the polypeptide comprises use of a small inhibitory RNA (siRNA) that specifically binds a polynucleotide encoding the polypeptide.

15. The method according to claim 12, wherein altering the level of the polypeptide comprises introducing into a cell a small inhibitory RNA (siRNA) that specifically binds a polynucleotide encoding the polypeptide.

16. The method according to claim 15, wherein the small inhibitory RNA is transcribed outside the cell and subsequently introduced into the cell.

17. The method according to claim 15, wherein the small inhibitory RNA is encoded in an expression plasmid introduced into the cell wherein the small inhibitory RNA is subsequently transcribed in the cell.

18. An antibody or fragment thereof that specifically binds the polypeptide according to claim 1.

19. The antibody or fragment according to claim 18, wherein the antibody or fragment is comprised in a kit, the kit further comprising information pertaining to the antibody.

20. The antibody according to claim 18, wherein the antibody is selected from the group consisting of a monoclonal antibody and a polyclonal antibody.

21. A method of screening agents that affect cell proliferation, the method comprising:
contacting candidate agents with at least one polypeptide having an amino acid sequence at least 85% homologous to the amino acid sequence selected from the group consisting of: SEQ ID NO: 2 and 4, and
evaluating the binding of the contacting against controls.


SEQUENCE LISTING (59 sequences)

SEQ ID NO: 1 - DNA, 1749 bp, Homo sapiens
atgatgaaacttagacacaaaaataaaaagccaggtgaaggttccaagggccacaagaagataagttggccctaccctcagcctgcaaagcaaaatgggaagaaagcaacctccaaagtgccctctgcacctcattttgttcaccccaatgatcatgccaatcgagaggctgaattaaagaagaagtgggttgaggagatgagggagaagcagcaagccgcccgggagcaagaaagacaaaaacgcaggaccattgagagctactgtcaggatgtcctaagacgccaggaggagtttgagcataaggaggaagttttgcaggaattaaatatgtttcctcagctggatgacgaggccacgaggaaggcttattacaaggagttccgtaaggtggtggaatactctgatgtgattctggaagtcctggatgccagagacccattaggctgccgctgcttccaaatggaggaggctgtcctgcgagcacaaggcaacaagaagctggtcctggtcttgaacaagattgacctggtccccaaggaggttgtggagaaatggctggattaccttcggaatgagttgccaaccgtggctttcaaggccagtacccagcatcaggtcaaaaacctgaatcgttgcagtgtgccagtagatcaggcctctgagtcactgctgaaaagcaaagcctgctttggagctgaaaacctcatgagggttctggggaactattgccgccttggtgaagtgcgcacccacattcgtgtgggtgttgtgggtcttcccaatgttgggaagagcagcctgatcaatagcctgaagcgcagccgcgcatgcagcgtgggagctgttcctggaattaccaaattcatgcaggaggtctacctggacaagttcatccggctcttggatgctccaggcattgtcccagggcccaactcagaggtgggcaccatcctgcgtaactgcgtccacgtgcagaagctggcagaccctgtgaccccagtggagaccatcctgcagcgctgcaacctggaggagatttccaactattatggcgtctctgggttccagaccactgagcactttctgacggcagtggcccaccgtttggggaagaagaagaagggaggcttatatagtcaggaacaggcggccaaagctgtcctagctgactgggtgagcgggaagatcagcttctatataccaccaccagccactcacactctgcccacccatctcagtgctgagatcgttaaggaaatgaccgaggtctttgacatcgaggatactgagcaggccaatgaagacaccatggaatgcttggccaccggagaatctgatgagctgttgggtgacacggacccacttgaaatggagatcaagttgctccattctccgatgacgaaaatagcagatgccattgaaaataaaaccaccgtgtataagattggagatctcactgggtattgcaccaatccaaaccgtcatcagatggggtgggctaaacgcaatgtggaccaccgccctaagagcaacagtatggtggatgtctgctcagtggaccgccgctcagtgctgcagaggatcatggagacggaccccctgcaacagggccaggctctggcatctgccctgaaaaataagaagaagatgcagaaacgtgcagataaaatcgccagcaagctgtctgattccatgatgtctgctctcgacctctctggcaatgctgatgatggtgttggtgactaa

SEQ ID NO: 2 - protein, 582 aa, Homo sapiens
MMKLRHKNKKPGEGSKGHKKISWPYPQPAKQNGKKATSKVPSAPHFVHPNDHANREAELKKKWVEEMREKQQAAREQERQKRRTIESYCQDVLRRQEEFEHKEEVLQELNMFPQLDDEATRKAYYKEFRKVVEYSDVILEVLDARDPLGCRCFQMEEAVLRAQGNKKLVLVLNKIDLVPKEVVEKWLDYLRNELPTVAFKASTQHQVKNLNRCSVPVDQASESLLKSKACFGAENLMRVLGNYCRLGEVRTHIRVGVVGLPNVGKSSLINSLKRSRACSVGAVPGITKFMQEVYLDKFIRLLDAPGIVPGPNSEVGTILRNCVHVQKLADPVTPVETILQRCNLEEISNYYGVSGFQTTEHFLTAVAHRLGKKKKGGLYSQEQAAKAVLADWVSGKISFYIPPPATHTLPTHLSAEIVKEMTEVFDIEDTEQANEDTMECLATGESDELLGDTDPLEMEIKLLHSPMTKIADAIENKTTVYKIGDLTGYCTNPNRHQMGWAKRNVDHRPKSNSMVDVCSVDRRSVLQRIMETDPLQQGQALASALKNKKKMQKRADKIASKLSDSMMSALDLSGNADDGVGD

SEQ ID NO: 3 - DNA, 1413 bp, Schizosaccharomyces pombe
atggtttccttaaaaaaaaagagtaaaagaagaactactcgtcttcgtagcagaattgaaaagaaagctgcagagagcaaacgcaagcaaaaaagagctgataaaaaaaatccgcaatggaaaagcagaattcctaaagatccaggtattcctaactcattcccctacaaggataaaattcttgctgagatcgaagaacagaagcgcattagagaagaagaaaaattggctcgtcgtgcttcaggacaagttgatgctgcaatggaagaggaagacgctgttgatgaaaatggatccctaatgatttcaaagatagctgaggctgcacaagcgtctaacccagatgatgaggaagagtttgtgatggaagaagacaaccttggagaggcccctttattagttgactccgagtcttatgaggcatcagtgaaggcagatacctctagaaaggcatatgataaggagttcaaaaaagttgttgaagcgtcagatgtgattctttacgttcttgacgctcgtgatcctgaagggactcgttcaaaagacgttgaacgtcaagttttagcatcttctgctgaagaaaagcgtcttattttcgttatcaacaaaattgacttagtaccatcagaagtactcaacaagtgggttacctatctccgcaatttctttcctacgattcctatgcgttccgcttcaggatctggaaactcaaatttaaagcaccagtcggcttctgctagttccactatctccaatcttcttaaatcactcaagtcttactcagccaagaagaagcttaaaagctctcttacagtcggtgtaattggataccccaacgttggtaaatcatctgttattaacgctcttgtcaaccgttctgcgaatggtcgttctgcaccttgtcctgccggaaacgttgccggaatgactacttctttgcgagaggtgaagctagataacaaattacgtttggtcgatagtcctggtattgtttttccttctagtgattctaaagacgatttgtatcgtcttgtaatgcttaatgctgtttcatccaccaaagttgatgatcccgttgccgttgcttcttacattttgcaattccttagcagagttccaggacaattggaaagaatgtttcaaagatatgagcttcctcctctattgaatacatccgatatagacactgctactgattttttagtcaatattgctcgtaagcgtggtcgtctaggtcgcggtggaattccaaatcttaacgctgctgccaatattgtcattaatgactggcatgctggtcgtatcgaatggtgggccgagcctgaggttataaacgaaaagaactcatctgaagttcaggatactcaaatcgttactgagtgggccaaagaatttgaccttaatgatttttga

SEQ ID NO: 4 - protein, 470 aa, Schizosaccharomyces pombe
MVSLKKKSKRRTTRLRSRIEKKAAESKRKQKRADKKNPQWKSRIPKDPGIPNSFPYKDKILAEIEEQKRIREEEKLARRASGQVDAAMEEEDAVDENGSLMISKIAEAAQASNPDDEEEFVMEEDNLGEAPLLVDSESYEASVKADTSRKAYDKEFKKVVEASDVILYVLDARDPEGTRSKDVERQVLASSAEEKRLIFVINKIDLVPSEVLNKWVTYLRNFFPTIPMRSASGSGNSNLKHQSASASSTISNLLKSLKSYSAKKKLKSSLTVGVIGYPNVGKSSVINALVNRSANGRSAPCPAGNVAGMTTSLREVKLDNKLRLVDSPGIVFPSSDSKDDLYRLVMLNAVSSTKVDDPVAVASYILQFLSRVPGQLERMFQRYELPPLLNTSDIDTATDFLVNIARKRGRLGRGGIPNLNAAANIVINDWHAGRIEWWAEPEVINEKNSSEVQDTQIVTEWAKEFDLNDF

SEQ ID NO: 5 - DNA, 132 bp, Artificial
  NB110 F
ggttaaaaaagaataatcggtaatgttttttctctagacaaccaactgtaaaatttgtaactacagcattttttacaatgcaacagctatgaccggctaccattcacccgctcaaccctcactaaagggaac

SEQ ID NO: 6 - DNA, 134 bp, Artificial
  NB111 R
gaaaaaccgcaaccgaaaaccaaatcccaaaatataagctctaagcaacaatagcttttttcgtaagttgaaaactctcattgtaaaacgacggcccgttctgccgagcatgacgacactatagggcgaattgg

SEQ ID NO: 7 - DNA, 96 bp, Artificial
  NB373 F
gcattgctaaactaaggaaatctttctaaatgtgaatataaattactaattagcttcaactttaaaaataacgagggaattcgagctcgtttaaac

SEQ ID NO: 8 - DNA, 91 bp, Artificial
  NB375 R
gttcttcttttactctttttttcttaagaaataagttagaaatagttacgcgtgcatatacttacttaaggaaactttgtatagttcatcc

SEQ ID NO: 9 - DNA, 33 bp, Artificial
  NB377 R
catctgcggccgcggaaaatcattaaggtcaaa

SEQ ID NO: 10 - DNA, 36 bp, Artificial
  NB378 F
cttacagtcggtgtaatttctgttattaacgctctt

SEQ ID NO: 11 - DNA, 36 bp, Artificial
  NB379 R
aagagcgttaataacagaaattacaccgactgtaag

SEQ ID NO: 12 - DNA, 44 bp, Artificial
  NB380 F
catatgtcgactatggtttccttaaaaaaaaagagtaaaagaag

SEQ ID NO: 13 - DNA, 37 bp, Artificial
  NB453 F
gatcgaagaacagaagcgcgaagacgctgttgatgaa

SEQ ID NO: 14 - DNA, 37 bp, Artificial
  NB454 F
ttcatcaacagcgtcttcgcgcttctgttcttcgatc

SEQ ID NO: 15 - DNA, 44 bp, Artificial
  NB455 F
gctactgattttttagtcaatattattccaaatcttaacgctgc

SEQ ID NO: 16 - DNA, 44 bp, Artificial
  NB456 R
gcagcgttaagatttggaataatattgactaaaaaatcagtagc

SEQ ID NO: 17 - DNA, 36 bp, Artificial
  NB457 F
aagttgttgaagcgtcagaagggactcgttcaaaag

SEQ ID NO: 18 - DNA, 36 bp, NB458 R
cttttgaacgagtcccttctgacgcttcaacaactt

SEQ ID NO: 19 - DNA, 36 bp, Artificial
  NB524 F
gcatcttctgctgaagaatcagaagtactcaacaag

SEQ ID NO: 20 - DNA, 36 bp, Artificial
  NB525 R
cttgttgagtacttctgattcttcagcagaagatgc

SEQ ID NO: 21 - DNA, 20 bp, Artificial
  NB629 R
cccaaaaagttaaaagatgg

SEQ ID NO: 22 - DNA, 17 bp, Artificial
  NB631 R
tcgttcaacacctcatc

SEQ ID NO: 23 - DNA, 18 bp, Artificial
  NB700 R
tcgttagaggtgagacaa

SEQ ID NO: 24 - DNA, 18 bp, Artificial
  NB702 F
agaagtggaaaaggagac

SEQ ID NO: 25 - DNA, 35 bp, Artificial
  NB712 F
catatgtcgactatggtgaagcccaagtacaaagg

SEQ ID NO: 26 - DNA, 34 bp, Artificial
  NB713 R
catctgcggccgcggctgcttttgtctgaatttt

SEQ ID NO: 27 - DNA, 32 bp, Artificial
  NB762 F
catatgtcgactatgatgaaacttagacacaa

SEQ ID NO: 28 - DNA, 36 bp, Artificial
  NB763 R
catctgcggccgcgggtcaccaacaccatcatcagc

SEQ ID NO: 29 - DNA, 33 bp, Artificial
  NB864 F
gactcaggtaccatggtttccttaaaaaaaaag

SEQ ID NO: 30 - DNA, 29 bp, Artificial
  NB865 R
gactcactcgagctatttgtatagttcat

SEQ ID NO: 31 - DNA, 32 bp, Artificial
  NB833 F
gactcaggtaccatgatgaaacttagacacaa

SEQ ID NO: 32 - DNA, 67 bp, Artificial
  NB907 F
gatccgctattatggcgtctctgggttcaagagacccagagacgccataatagcttttttggtaccg

SEQ ID NO: 33 - DNA, 67 bp, Artificial
  NB908 R
aattcggtaccaaaaaagctattatggcgtctctgggtctcttgaacccagagacgccataatagcg

SEQ ID NO: 34 - DNA, 67 bp, Artificial
  NB909 F
gatccgctatattgcggtctggtcgttcaagagacgaccagaccgcaatatagcttttttggtaccg

SEQ ID NO: 35 - DNA, 67 bp, Artificial
  NB910 R
aattcggtaccaaaaaagctatattgcggtctggtcgtctcttgaacgaccagaccgcaatatagcg

SEQ ID NO: 36 - DNA, 39 bp, Artificial
  NB963 F
ggccgcagatggactacaaggatgacgatgacaaataac

SEQ ID NO: 37 - DNA, 39 bp, Artificial
  NB964 R
tcgagttatttgtcatcgtcatccttgtagtccatctgc

SEQ ID NO: 38 - DNA, 96 bp, Artificial
  NB1002 F
gttaaaaaagaataatcggtaatgttttttctctagacaaccaactgtaaaatttgtaactacagcattttttacaatgatgaaacttagacacaa

SEQ ID NO: 39 - DNA, 97 bp, Artificial
  NB1004 F
cgaaaagaactcatctgaagttcaggatactcaaatcgttactgagtgggccaaagaatttgaccttaatgattttccgcggccgcagatggactac

SEQ ID NO: 40 - DNA, 36 bp, Artificial
  NB1006 F
aacaaattacgtttggtcattgtttttccttctagt

SEQ ID NO: 41 - DNA, 36 bp, Artificial
  NB1007 R
actagaaggaaaaacaatgaccaaacgtaatttgtt

SEQ ID NO: 42 - DNA, 18 bp, Artificial
  NB1102 F
gatataattaattcagac

SEQ ID NO: 43 - DNA, 92 bp, Artificial
  NB1125 R
aaccgcaaccgaaaaccaaatcccaaaatataagctctaagcaacaatagcttttttcgtaagttgaaaactctctagaactagtggatctg

SEQ ID NO: 44 - DNA, 49 bp, Artificial
  NB1149 F
gacgcaggtaccgtcgacgagctcctttatattaaaaattattaattgc

SEQ ID NO: 45 - DNA, 28 bp, Artificial
  NB1150 R
gctatctttgaaatcattagggatccat

SEQ ID NO: 46 - DNA, 38 bp, Artificial
  NB1151 F
actacagcattttttacaatggtttccttaaaagctgc

SEQ ID NO: 47 - DNA, 38 bp, Artificial
  NB1152 R
gcagcttttaaggaaaccattgtaaaaaatgctgtagt

SEQ ID NO: 48 - DNA, 40 bp, Artificial
  NB1153 F
actacagcattttttacaatggtttccttaaaaaatccgc

SEQ ID NO: 49 - DNA, 40 bp, Artificial
  NB1154 R
gcggattttttaaggaaaccattgtaaaaaatgctgtagt

SEQ ID NO: 50 - DNA, 33 bp, Artificial
  NB1160 F
catatgtcgactatgaaaaggcctaagttaaag

SEQ ID NO: 51 - DNA, 36 bp, Artificial
  NB1161 R
catctgcggccgcggcacataatctgtactgaagtc

SEQ ID NO: 52 - DNA, 16 bp, Artificial
  NB1478 F
tttcgctgcgttcttc

SEQ ID NO: 53 - DNA, 32 bp, Artificial
  FLAG sequence
agatggactacaaggatgacgatgacaaataa

SEQ ID NO: 54 - DNA, 19 bp, Artificial
  For siRNA in human GNL3L (target sequence)
ctattatggcgtctctggg

SEQ ID NO: 55 - DNA, 19 bp, Artificial
  For siRNA knockdown of human GNL3L (scrambled seq)
ctatattgcggtctggtcg

SEQ ID NO: 56 - DNA, 44 bp, Artificial
  Human GNL3L forward
atgtgcgaattcatgatgaaacttagacacaaaaataaaaagcc

SEQ ID NO: 57 - DNA, 36 bp, Artificial
  Human GNL3L reverse
caccatgatatcccggatgaacttgtccaggtagac

SEQ ID NO: 58 - DNA, 16 bp, Artificial
  Human beta-actin forward
ggcgacgaggcccaga

SEQ ID NO: 59 - DNA, 16 bp, Artificial
  Human beta-actin reverse
cgatttcccgctcggc